(19) United States
(12) Patent Application Publication
(10) Pub. No.: US 2020/0268968 A1
(43) Pub. Date: Aug. 27, 2020

(54) METHODS AND SYSTEMS FOR MANAGING DIABETES

(71) Applicants: Children`s Medical Center Corporation, Boston, MA (US); Joslin Diabetes Center, Inc., Boston, MA (US)

(72) Inventors: Gary Steil, Boston, MA (US); Howard Wolpert, Brookline, MA (US); Michael Agus, Newton, MA (US); Paulina Ortiz-Rubio, Brookline, MA (US)

(21) Appl. No.: 16/095,195

(22) PCT Filed: Apr. 21, 2017
(86) PCT No.: PCT/US2017/028860

§ 371 Date: Oct. 19, 2018

Related U.S. Application Data
(60) Provisional application No. 62/326,496, filed on Apr. 22, 2016.

(51) Int. Cl. A61M 5/172; A61B 5/145; A61B 5/00; A61B 5/11; A61M 5/142; G16H 10/60; G16H 20/17; G16H 40/63

(52) U.S. Cl. CPC A61M 5/1723; A61B 5/14532; A61B 5/4839; A61B 5/7275; A61B 5/1118; A61M 2230/201; G16H 10/60; G16H 20/17; G16H 40/63; A61M 2205/502; A61M 5/142

(57) ABSTRACT

This disclosure relates to systems and methods for diabetes management.

### CLAIM OF PRIORITY

This application claims the benefit of U.S. Provisional Application Ser. No. 62/326,496, filed on Apr. 22, 2016. The entire contents of the foregoing are incorporated herein by reference.

### TECHNICAL FIELD

This disclosure relates to diabetes management.

### BACKGROUND

Diabetes mellitus is a prevalent and degenerative disease characterized by insulin deficiency, which prevents normal regulation of blood glucose levels leading to hyperglycemia and ketoacidosis.

Insulin promotes glucose utilization, protein synthesis, formation and storage of neutral lipids, and the growth of some cell types. Insulin is produced by the 3 cells within the islets of Langerhans of the pancreas. Traditionally, insulin has been injected with a syringe. More recently, use of insulin pump therapy has been increasing, especially for delivering insulin for diabetics. However, insulin pumps can be limited in their ability to replicate all of the functions of the pancreas. Thus, there is a considerable interest to improve the pump to better simulate the function of a pancreas.

### SUMMARY

This disclosure relates to a Clinical Decision Support (CDS) system for diabetes management. The CDS system determines a blood glucose level and/or makes a recommendation to an insulin pump parameter based on a plurality of data records representing one or more predicting factors, e.g., activity data, nutritional information, past blood glucose levels, the rate of change of blood glucose level and/or other contextual data.

In one aspect, the disclosure relates to a computer-implemented method of predicting a blood glucose level of a subject. The method includes: receiving and storing a plurality of historical data records representing one or more predicting factors of the subject and a corresponding blood glucose level of the subject for a past period of time; inputting into a data processing engine the plurality of historical data records, and determining a set of parameters corresponding to the historical data records; inputting into the data processing engine the set of parameters and a current data record representing one or more predicting factors of the subject, thereby predicting a blood glucose level of the subject corresponding to the current data record; and outputting information indicative of the predicted blood glucose level corresponding to the current data record.

In some embodiments, the blood glucose level is nighttime nadir glucose (NNG), morning fasting glucose (MFG), 2-hour postprandial glucose (PPG2HR), 5-hour postprandial glucose (PPG5HR), or 5 hour nadir postprandial glucose (NPP5HR).

In some embodiments, the historical data records representing one or more predicting factors include a data record of a level of physical activity. In some embodiments, the level of physical activity is measured by a continuous activity monitor.

In some embodiments, the historical data records representing one or more predicting factors include a data record of the fat content of a meal and/or the carbohydrate content of a meal. In some embodiments, the historical data records representing one or more predicting factors include a data record of the blood glucose level of the subject at a time point. In some embodiments, the historical data records representing one or more predicting factors include a data record of a rate of change of a blood glucose level over a specific time interval. In some embodiments, the historical data records representing one or more predicting factors include historical data records that are observed over a prior window of time.

In some embodiments, the data processing engine determines the parameters based on historical data records that are received within the fixed moving time window. In some embodiments, during the step of determining the parameters, the data processing engine gives less weight to historical data records that received at points further in the past with a forgetting factor configured to define how long in the past before weight becomes equal to e⁻¹. In some embodiments, the fixed moving time window is 1 month, 3 months, 6 months, or 12 months.

In some embodiments, the method further includes the step of sending an alert to the subject or the subject's caregiver when the blood glucose level of the subject for the time interval of interest is outside a predetermined range. In some embodiments, the method further includes the step of adjusting an insulin pump for the subject upon receiving the alert.

The disclosure also relates to a computer-implemented method of making a therapy recommendation for an insulin pump parameter. The method includes receiving a blood glucose level at a first time point; receiving a rate of change of the blood glucose level at a second time point; determining an adjusted value for an insulin pump parameter based on the blood glucose level at the first time point and the rate of change of the blood glucose level at the second time point; and making a therapy recommendation for an insulin pump parameter based on the adjusted value. In some embodiments, the first time point and the second time point is the same time point.

In some embodiments, the insulin pump parameter is a basal rate for a time window. In some embodiments, the basal rate in time windows is from 12:00 AM to 1:00 AM, from 1:00 AM to 2:00 AM, from 2:00 AM to 3:00 AM, from 3:00 AM to 4:00 AM, from 4:00 AM to 5:00 AM, from 5:00 AM to 6:00 AM, from 6:00 AM to 7:00 AM, from 7:00 AM to 8:00 AM, from 8:00 AM to 9:00 AM, from 9:00 AM to 10:00 AM, from 10:00 AM to 11:00 AM, from 11:00 AM to 12:00 PM, 12:00 PM to 1:00 PM, from 1:00 PM to 2:00 PM, from 2:00 PM to 3:00 PM, from 3:00 PM to 4:00 PM, from 4:00 PM to 5:00 PM, from 5:00 PM to 6:00 PM, from 6:00 PM to 7:00 PM, from 7:00 PM to 8:00 PM, from 8:00 PM to 9:00 PM, from 9:00 PM to 10:00 PM, from 10:00 PM to 11:00 PM, or from 11:00 PM to 12:00 AM.

In some embodiments, the adjusted value for the insulin pump parameter is determined by comparing the rate of change of the blood glucose level to a desired rate of change of the blood glucose level.

In some embodiments, an insulin pump parameter is modulated when the difference between the adjusted value for the insulin pump parameter and the parameter that is in use is greater than a pre-determined threshold. In some embodiments, the insulin pump parameter is modulated for a portion of the difference between the adjusted value for the insulin pump parameter and the parameter that is in use, wherein the portion is ⅕, ¼, ⅓, or ½.

In some embodiments, the insulin pump parameter is a bolus estimation (BE). In some embodiments, the bolus estimation is determined by comparing the rate of change of blood glucose level at a time point to a desired rate of change of blood glucose level at the same time point. In some embodiments, the bolus estimation is determined by further taking into account insulin on board (IOB). In some embodiments, the bolus estimation is determined by furthering taking into account fat content in a meal. In some embodiments, the bolus estimation is a meal bolus. In some embodiments, the bolus estimation is determined by further taking into account the interaction between fat content and carbohydrate content.

The present disclosure also relates to a computer-implemented method of adjusting an insulin pump parameter. The method includes: sending a plurality of data records representing one or more predicting factors of the subject to a server through a network; receiving an adjusted value for an insulin pump parameter from the server, wherein the adjusted value for an insulin pump parameter is determined by the plurality of data records representing the one or more predicting factors; and modulating the insulin pump parameter based on the adjusted value. In some embodiments, the insulin pump parameter is a basal rate, a bolus estimation, carbohydrate to insulin ratio (CIR), and/or Insulin Sensitivity Factor (ISF). In some embodiments, the insulin pump parameter is a basal rate for a period of time. In some embodiments, the plurality of data records representing one or more predicting factors include a level of physical activity of the subject, a fat content of a meal take by the subject, a carbohydrate content of a meal taken by the subject, a blood glucose level of the subject at a time point, and/or a rate of change of blood glucose level of the subject at a time point. In some embodiments, the insulin pump parameter is modulated for a portion of the difference between the adjusted value for the insulin pump parameter and the parameter that is in use, wherein the portion is ⅕, ¼, ⅓, or ½.

The present disclosure provides several advantages. First, the parameters of the CDS algorithms are determined based on data records for each individual patient. Thus, the CDS system can account for variations among different individuals, and tailor the CDS algorithm for each individual patient. Second, the CDS system takes into account the rate of change of the blood glucose level over time and the rate of change of insulin-on-board and not just specific values of these parameters at a given point in time. This allows the CDS system to adjust for pharmacokinetic/pharmacodynamics delays. Third, the CDS system determines insulin dosing patterns based on different nutritional components of a meal, and how the nutritional components interact with each other, whereas many existing bolus calculators rely almost exclusively on carbohydrate content. Fourth, the CDS system provides an integrated approach for diabetes management by storing and processing data records of a patient in a server, thereby facilitating diabetes management for care givers and patients.

As used herein, the term “predicting factor” refers to a quantifiable variable that is used in a CDS algorithm. Predicting factors typically have some influences on or have relationships with the outcome of a CDS algorithm, and thus can be used in a CDS algorithm to determine the value of the outcome. Examples of predicting factors include, but are not limited to, a level of physical activity, blood glucose levels at various time points, a rate of change of blood glucose level at various time points, fat content in a meal, carbohydrate content in a meal, and interaction terms between these predictors. In some instances the outcome of the CDS algorithm is to provide a recommended change in insulin dosing to the physician (e.g. a recommendation to change a basal rate, CIR, or ISF); in other instances, the recommendation is provided to the patient (e.g., sending a physician approved text message to the patient at 8 PM telling them they should change their 8 PM to 6 AM basal profile for that night in response to high activity or other predictor of nighttime hypoglycemia).

As used herein, the term “parameter” refers to a numerical or other measurable factor forming one of a set that defines a system or sets the conditions of its operation. For the data processing engines configured to execute CDS algorithms, parameters include, but are not limited to, expected (mean) value, coefficients, thresholds, proportional forms (k), integration time, etc.

As used herein, the term “historical data record” refers to a data record that was collected before the time of interest such as the current time, i.e., a data record that was collected at least 12 hours before the time of interest. The historical data records can be used by a data processing engine to determine appropriate parameters. In some instances the historical record may include a weighted history, or moving average of several weeks of data, where as in other cases the history may only include data obtained on the day in question. For example, the CDS system may need several weeks of data before concluding that daytime activity is a significant predictor of nighttime hypoglycemia at which time it would recommend to the physician or other care provider a new basal rate for use during nights following high activity. Thereafter, the CDS system may send notifications to the patient based only on an activity record comprised only of the activity recorded on the day in questions (e.g., step count from midnight to 8 PM). Historical data records can be collected more than 12 hours before the time of interest, e.g., 1 day before the time of interest, 2 days before the time of interest, 1 week before the time of interest, and 1 month before the time of interest.

As used herein, the term “current data record” refers to a data record that is collected at or near the time of interest, e.g., the current time, i.e., a data record that was collected in the past 48 hours, in the past 36 hours, in the past 24 hours, in the past 12 hours. In some embodiments, the current time frame is limited to 24-48 hours.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

The disclosure contains at least one drawing executed in color. Copies of this patent or patent application publication with color drawing(s) will be provided by the Office upon request and payment of the necessary fee.

FIG. 1 is a diagram illustrating one exemplary Clinical Decision Support (CDS) system.

FIG. 2 is a flow diagram of an exemplary process of the CDS system to make a therapy recommendation to adjust an insulin parameter.

FIG. 3a is a graph showing night basal adaptation before CDS adaption over 24 hours from about 7 am to 7 am. The top panel shows the starting nighttime basal rates for a 7 year old boy and the lower panel shows the corresponding glucose level as determined by continuous glucose monitoring (CGM). The solid triangles along the bottom indicate times of use of supplemental carbohydrate to prevent or correct hypoglycemia.

FIG. 3b is a graph showing night basal adaptation of the subject in FIG. 3a following CDS over 24 hours from about 7 am to 7 am in which activity (Low activity, LA; high activity, HA) is identified as a predictor of nighttime nadir glucose. Activity is measured as FitBit® step count at 6 PM.

FIG. 4a is a graph showing Low (LF) and high fat (HF) meal response at start of CDS. Controlled study in adults with type 1 diabetes. Fitted lines are from a low-order identifiable metabolic model.

FIG. 4b is a graph showing Low (LF) and high fat (HF) meal response following ˜6 weeks of CDS. Controlled study in adults with type 1 diabetes. Fitted lines are from a low-order identifiable metabolic model.

FIG. 5a is a graph showing 2 U bolus was given to a subject at the time point TBOLUS.

FIG. 5b is a graph showing insulin on board (IOB) for typical (Blue) and Medtronic (Red) Pumps assuming an IOB hour half-life of 2 hours.

FIG. 6a is a graph showing insulin concentration (closed red circles) and effect (glucose infusion to maintain euglycemia; closed green circles) with 3 compartment PK/PD model fit (subcutaneous depot, plasma, and remote compartment interstitial fluid compartment surrounding insulin sensitive tissue).

FIG. 6b is a panel of three graphs showing PK/PD and IOB profile for a 3.95 U insulin bolus given at 1 am.

FIG. 6c is a panel of three graphs showing PK/PD and IOB profile for a 1.16 U bolus given at 3 am.

FIG. 6d is a graph showing IOB profiles superimposed from 4 am.

FIG. 7 is a graph showing blood glucose concentrations over 6 hours in 10 adults with type 1 diabetes following a low fat, low protein (LFLP) meal and a high fat, high protein (HFHP) meal with insulin dosed using the individualized carbohydrate:insulin ratio, and the same HFHP with an adjusted insulin dose using a model predictive bolus. Dashed line indicates target fasting glucose of 126 mg/dL (impaired fasting glucose threshold).

FIG. 8a is a graph showing comparison of baseline glucose levels for the HFHP, LFHP and HFHPMPB groups. P value indicates ANOVA with post-hoc comparison value corrected for multiple comparisons.

FIG. 8b is a graph showing comparison of postprandial AUC for the HFHP, LFHP and HFHPMPB groups. P value indicates ANOVA with post-hoc comparison value corrected for multiple comparisons.

FIG. 8c is a graph showing comparison of peak postprandial blood glucose levels for the HFHP, LFHP and HFHPMPB groups. P value indicates ANOVA with post-hoc comparison value corrected for multiple comparisons.

FIG. 8d is a graph showing comparison of two-hour postprandial blood glucose levels for the HFHP, LFHP and HFHPMPB groups. P value indicates ANOVA with post-hoc comparison value corrected for multiple comparisons.

FIG. 9 is a graph showing comparison of blood glucose levels after consuming a pizza without cheese (labeled low fat low protein or LFLP) and with cheese (labeled high fat high protein or HFHP) in 10 individuals with type 1 diabetes.

FIG. 10a is a graph showing an insulin bolus with DOSE (U) calculated from an individuals' standard CIR (red shaded area) with 50% of the dose given immediately and 50% given over a DURATION of 2 hours; blue shaded area shows the insulin bolus after optimization for total DOSE (U), % of DOSE given immediately, and DURATION.

FIG. 10b is a graph showing inappropriate blood glucose (BG) profile (open circles) obtained with individuals standard CIR (as shown in FIG. 10a red shaded area). Model fit of same data (red line). Model predicted fit with optimized bolus (blue line; optimal bolus as shown in FIG. 10a blue shaded area). And, meal blood glucose response obtained on repeating the same meal (blue closed circles). Metabolic model was used to fit BG profile obtained with standard bolus, and predict glucose response to optimized bolus (as shown in FIG. 10c).

FIG. 10c is a schematic diagram of low order identifiable metabolic showing how blood glucose profile (G) changes in response to pump insulin deliver (PUMP_{ID}) and meal rate of glucose appearance (RA_{[MEAL]}). RA_{[MEAL]} (green shaded area) is shown as a piecewise continuous profile characterized by an initial rise to maximal vale, fixed time at maximal value, and linear decrease to zero. Compartments representing the pump insulin delivery site (I_{SC}), plasma insulin (I_{P}), and remote interstitial fluid (ISF) surrounding insulin sensitive tissue (typically fat and muscle) are shown as circles. Compartment representing glucose concentration in plasma and tissues that rapidly equilibrate with plasma (liver and splanchnic bed) are represented as G. Endogenous glucose appearance (primarily hepatic) is represented as R_{A[ENDO]} (insulin sensitive). Optimal model predicted bolus (MPB) is obtained in two steps: first, parameters of the model are identified by choosing parameters of the model to minimize the squared difference in model prediction and observed blood glucose response (non-linear least squares). Second, using the model and parameters identified in step 1, the PUMP_{ID} profile is chosen to minimize a predefined cost function (typically sum of differences between predicted glucose and target glucose).

FIG. 11 is a graph showing simulation results for observed Peak Post Prandial (PPP) glucose divided by Target PPP (ratio of 1 being ideal). Observed PPP is assumed to be affected by CIR, but with a substantial component due to unexplained variance (normally distributed mean 0, standard deviation 1). Target PPP is assumed to linearly increase with size of meal (also randomly chosen but with uniform distribution). Individual points are for individual meals; black solid line is a moving smoothed average. CIR (FIG. 12) adapts over several months to achieve the desired ratio of 1.

FIG. 12 is a graph showing time course of changes to CIR as determined by Eq. 6b. Time course shows CIR converges to a value that leads to the desired PPP glucose response (ratio of observed PPP to Target PPP shown FIG. 11) over a couple of months (200 meals).

### DETAILED DESCRIPTION

Insulin pump therapy (IPT) combined with continuous glucose monitoring (CGM), allows individuals with type 1 diabetes to better manage their blood glucose levels. However, the pumps still need to be configured with basal insulin delivery rates, carbohydrate to insulin ratios (CIR), glucose correction factors (GCF), and insulin-on-board (IOB) time profiles. Insulin requirements often vary between days depending on various factors (e.g., the history and type of food consumed and the amount of physical activity). Adjusting insulin delivery to account for these added nutritional and activity factors is challenging.

In some instances, the insulin pump can be set to provide one or more different basal insulin delivery rates during different time intervals of the day. These different basal rates at various time intervals during the day usually depend on a patient's lifestyle and insulin requirements. For example, many insulin pump users require a lower basal rate overnight while sleeping and a higher basal rate during the day, or users might want to lower the basal rate during the time of the day when they regularly exercise.

A bolus is an extra amount of insulin taken to cover a rise in blood glucose, often related to a meal or snack. Whereas a basal rate provides continuously pumped small quantities of insulin over a long period of time, a bolus provides a relatively large amount of insulin over a fairly short period of time. Most boluses can be broadly put into two categories: meal boluses and correction boluses. A meal bolus is the insulin needed to control the expected rise in glucose levels due to a meal. A correction bolus is the insulin used to control unexpected highs in glucose levels. Often a correction bolus is given at the same time as a meal bolus because patients often notice unexpected highs in glucose levels when preparing to deliver a meal bolus related to a meal.

Target Blood Glucose (Target) is the target blood glucose (BG) that the user would like to achieve and maintain. Specifically, a target blood glucose value is typically between 70-120 mg/dL for preprandial BG, and 100-150 mg/dL for postprandial BG.

Insulin Sensitivity Factor (ISF) is a value that reflects how far the user's blood glucose drops in milligrams per deciliter (mg/dl) when one unit of insulin is taken. An example of an ISF value is 1 Unit for a drop of 50 mg/dl, although ISF values will differ from user to user.

Carbohydrate-to-Insulin Ratio (CIR) is a value that reflects the amount of carbohydrates that are covered by one unit of insulin. An example of a CIR is 1 Unit of insulin for 15 grams of carbohydrates. Similarly, CIR values will differ from user to user.

Insulin Pump settings are typically adjusted by patients or by their physician. An example of an insulin pump can be found, e.g., in U.S. Pat. No. 6,554,798. Many of the insulin pump adjustments are made using incomplete “logbook data” (paper-based records maintained by the patient). In cases were CGM data are available, physicians rarely have sufficient time to review the data or combine it with pump or logbook data. This becomes more challenging in instances where patient is struggling to understand the subtleties of underlying the need to make acute adjustments, or instances where a parent may be adjusting a child's dose without knowledge of prior activity or food consumption as will happen when the child is at school or day-care. In many cases, therapy adjustments are made after too few observations. The described methods rely on statistical and engineering control theory to ensure a sufficient amount of data is acquired prior to making recommendations to alter insulin delivery and can reconstruct prior events using advanced metabolic models.

The present disclosure relates to a Clinical Decision Support (CDS) system and methods that use activity data, nutritional information, and other contextual data to guide day-to-day insulin dosing. The system obtains data from various sources, for example, activity data from Continuous Activity Monitors (e.g., FitBit® Activity Monitors), blood glucose level data from Continuous Blood Glucose Monitors, and nutritional information from meal apps (e.g., MyFitnessPal from a mobile phone). The systems can store the data in a server. In some embodiments, the described methods combine the data with CGM and pump data at regular intervals, up to once per day, allowing for an on-going analysis of trends in key glucose metrics, e.g., fasting glucose, 2-hour postprandial glucose, and incidence of hypoglycemia. It will alert the patient or responsible care provider of any conditions that might warrant intervention (e.g., reduce nighttime basal rate in response to high daytime activity) or any need to change in pump parameters (e.g., increase CIR ratio, make fixed adjustment in basal rate). To this end the described methods specifically incorporate dietary fat and alcohol intake into the adaptive monitoring as, in adults, these are major factors that contribute to variability in glucose control. In some embodiments, the described methods provide recommendations for adjustments in the alarm thresholds available with CGM devices (smart alarm). In some embodiments, the described methods can send an alert (e.g., an email, an alarm) to patients, or parents of younger patients, requesting additional information at some appropriate situation (e.g., following hypoglycemia). The described methods are largely transparent to the user, as each device (e.g., pump, CGM, activity monitor) is configured to synchronize with the cloud, for example, a device is synchronized with the cloud when the device is connected to a cellphone, tablet, or personal computer by Bluetooth.

The described methods also relate to Insulin Pump Therapy (IPT) and Multiple Daily Injection (MDI) therapy. The combination of statistical models and testing procedures can ensure each therapy recommendation is robust to normal day-to-day variability in managing an individual with diabetes.

### Clinical Decision Support (CDS) Systems

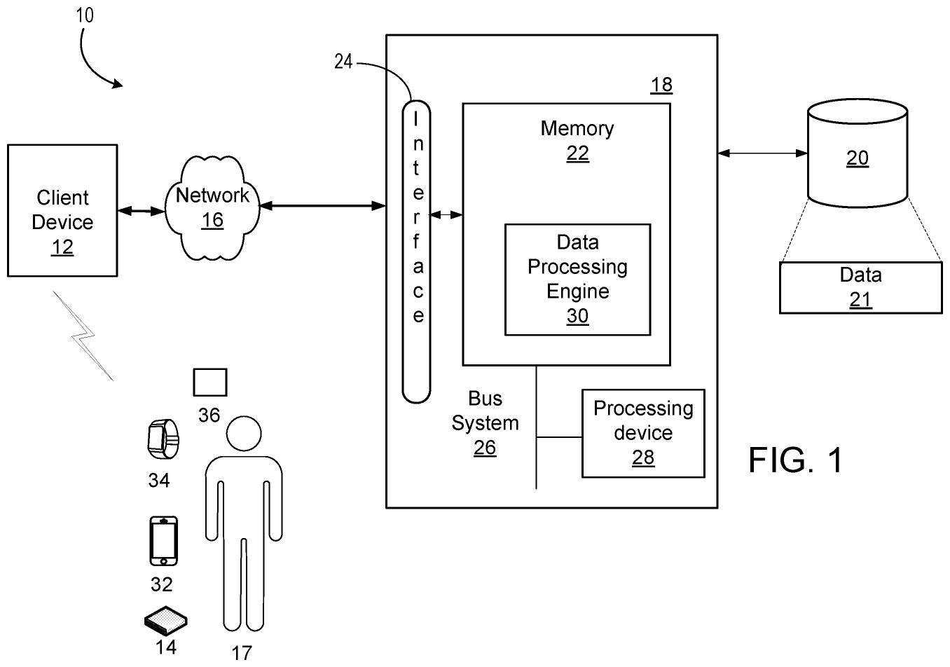

Referring to FIG. 1, system 10 collects data from various resources (e.g., activity monitor 34, blood glucose monitor 36, client device 32, insulin pump 14 etc.), stores data 21 in data repository 20, applies data processing engine 30 that implements various CDS algorithms to data 21, predicts various outcomes (e.g., fasting glucose, 2-hour postprandial glucose, and incidence of hypoglycemia), and makes a therapy recommendation for a parameter in insulin pump 14. System 10 also includes subject 17, client device 12, data processing system 18, network 16, interface 24, memory 22, bus system 26, and processing device 28.

System 10 collects data from various resources. In some embodiments, system 10 collects activity data of subject 17 from activity monitor 34 (e.g., Continuous Activity Monitors). In some embodiments, system 10 collects blood glucose level data from blood glucose monitor 36 (e.g., Continuous Blood Glucose Monitors). In some embodiments, system 10 collects nutritional information from meal apps (e.g., MyFitnessPal) from client device 32.

In some embodiments, activity monitor 34, blood glucose monitor 36, client device 32, and insulin pump 14 can communicate with client device 12 via various ways, e.g., Bluetooth, universal serial bus (USB) cable, wireless networking, etc.

Client device 12 and client device 32 can be any computing device capable of taking input from a user and communicating over network 16 with data processing system 18 and/or with other client devices. Client device 12 can be a mobile device, a desktop computer, a laptop, a cell phone, a personal digital assistant (PDA), a server, an embedded computing system, a mobile device and so forth. In some embodiments client device 12 and client device 32 are the same device.

Data processing system 18 receives data 21 from client device 12 via network 16. In some embodiments, data processing 18 stores data 21 in data repository 20. Data processing system 18 can retrieve, from data repository 20, data 21 representing a plurality of data records for CDS algorisms that are related to subject 17, e.g., activity, blood glucose level at various time intervals, blood glucose level change at various time point, meal contents etc.

Data processing system 18 inputs the retrieved data into memory 22. Data processing engine 30 is programmed to apply CDS algorithms to data 21. There are various types of CDS algorisms, including, but are not limited to, multivariate statistical model for predicting therapy adjustment (MSM-TA), multi-input-multi-output (MIMO) adaptive proportional integral derivative (APID) control algorithm (MIMO-APID), metabolic model, various algorithms for optimal bolus estimation etc.

The algorithm uses two separate time frames—current and historic. For example, in using activity to predict future insulin requirement the a recommendation may be sent to the patient at 8 PM to lower the basal rate that night (e.g., 8 PM to 6 AM the next morning) basal on the activity that has occurred that day (step count from midnight to 8 PM). In contrast, more subtle changes in insulin requirement may not become apparent until several months of data is acquired. For example, as the patient becomes older, losses or gains weight, or changes their diet. Under some conditions it may also take several weeks of data to establish an observation is statistically significant; for example, several months of data may be required to establish daytime activity significantly effects nighttime nadir glucose. Under these conditions the historic data may be a fixed moving window—perhaps 3 to 6 weeks depending on the magnitude of the effect and how often the patient exercises. In some embodiments, a recursive formulation may be used that effectively results in an infinite window; in other embodiments a forgetting factor may be introduced that gives exponentially less weight to data obtained further in the past. For example, setting the forgetting factor to 14 days would mean today's data gets weighted as one (e^{-0/14}); data that is 7 days old gets weighted 0.61 (e^{-7/14}), data that is 14 days old gets weighted 0.37 (e^{-14/14}) and data that is 28 days old gets weighted 0.14 (e^{-28/14}). In theory, this scheme is considered infinite in duration (e(^{-500/14} or e^{-1000/14} is still a finite number), but in practice data that is 6 weeks old begins to have no meaningful effect (e^{-6×7/14}=0.05). Generally, setting the forgetting factor to a large number makes the adaptation robust to noise or interday variability in the glucose values (i.e., limits the number of changes in a pump setting) but also limits the algorithms ability to rapidly respond to changing conditions.

In some embodiments, data processing engine 30 is configured to apply a multivariate statistical model for predicting therapy adjustment (MSM-TA). Data processing system 18 executes data processing engine 30, thereby the MSM-TA algorithm to data 21 representing appropriate predictors, e.g., subject 17's physiological conditions, blood glucose levels, daytime activity, meal fat content, etc. Based on application of data processing engine 30, data processing system 18 determines an outcome and outputs, e.g., to client device 12 via network 16, client device 32, and/or insulin pump 14, data indicative of the determined outcome. In some embodiments, the outcome can be blood glucose level, e.g., nighttime nadir glucose (NNG), morning fasting glucose (MFG), 2 and 5-hour postprandial glucose (PPG_{2HR} and PPG_{5HR}) and 5 hour nadir postprandial glucose (NPP_{5HR}), etc. In some embodiments, if the outcome falls outside a pre-determined range, client device 16, client device 32, and/or insulin pump 14 will generate/send an alert to appropriate individuals, e.g., subject 17 and/or the subject's caregiver. The appropriate individual will determine whether any intervention is necessary, e.g., by adjusting the parameter for the insulin pump, consuming additional food, administering urgent care, etc.

In some embodiments, data processing system 18 applies CDS algorithms only to data 21 that are collected within a window of time, for example, in the past one month, in the past two months, in the past three months, in the past 6 months, in the past year etc. In some embodiments, data processing system 18 applies CDS algorithms only to data 21 that are related to subject 17.

In some embodiments, data processing engine 30 is configured to apply various algorithms, e.g., Multi-Input-Multi-Output (MIMO) Adaptive Proportional Integral Derivative (APID) control algorithm (MIMO-APID) and optimal bolus estimation (OPT-BE) algorithm. Data processing system 18 executes data processing engine 30, thereby applying the algorithm to data 21. Based on application of data processing engine 30, data processing system 18 determines an outcome and outputs, e.g., to client device 12 via network 16, client device 32, and/or insulin pump 14, data indicative of the determined outcome. In some embodiments, the outcome can be an optimized value for an insulin parameter, e.g., basal rates from 12 am to 3 am, 3 am to 5 am, and 5 am to 7 am, or basal rates from 5 pm to 7 pm, 7 to 9 pm, and 9 to midnight, bolus, meal bolus, correction bolus, ISF, CIR, etc.

In some embodiments, when the recommended insulin parameter is higher than a predetermined threshold, data processing system 18 will communicate with insulin pump 14 via network 16 and client device 12, and sends the optimized value of an insulin pump parameter to insulin pump 14.

In some embodiments, data processing system 18 generates data for a graphical user interface that when rendered on a display device of client device 12 and/or client device 32, display a visual representation of the output.

In some embodiments, data processing system 18 sends data 21 and/or the outcome of data processing engine 30 to a third client device, which allows a subject's caregiver to review and determine whether any intervention or adjustment is necessary. In some embodiments, the values for the outcomes can be stored in data repository 20 or memory 22.

Data processing system 18 can be a variety of computing devices capable of receiving data and running one or more services. In one embodiment, data processing system 18 can include a server, a distributed computing system, a desktop computer, a laptop, a cell phone, a rack-mounted server, and the like. Data processing system 18 can be a single server or a group of servers that are at a same position or at different positions (i.e., locations). Data processing system 18 and client device 12 can run programs having a client-server relationship to each other. Although distinct modules are shown in the figures, in some embodiments, client and server programs can run on the same device.

Data processing system 18 can receive data from activity monitor 34, client device 32, blood glucose monitor 36, insulin pump 14, and/or client device 12 through input/output (I/O) interface 24, and data repository 20. Data repository 20 can store a variety of data values for data processing engine 30. The data processing engine (which may also be referred to as a program, software, a software application, a script, or code) can be written in any form of programming language, including compiled or interpreted languages, or declarative or procedural languages, and it can be deployed in any form, including as a stand-alone program or as a module, component, subroutine, or other unit suitable for use in a computing environment. The data processing engine may, but need not, correspond to a file in a file system. The program can be stored in a portion of a file that holds other programs or information (e.g., one or more scripts stored in a markup language document), in a single file dedicated to the program in question, or in multiple coordinated files (e.g., files that store one or more modules, sub programs, or portions of code). The data processing engine can be deployed to be executed on one computer or on multiple computers that are located at one site or distributed across multiple sites and interconnected by a communication network.

In one embodiment, data repository 20 stores data 21 indicative of various input values for CDS algorithms. In another embodiment, data repository 20 stores outcomes of CDS algorithms.

I/O interface 24 can be a type of interface capable of receiving data over a network, including, e.g., an Ethernet interface, a wireless networking interface, a fiber-optic networking interface, a modem, and so forth. Data processing system 18 also includes a processing device 28. As used herein, a “processing device” encompasses all kinds of apparatus, devices, and machines for processing information, including by way of example a programmable processor, a computer, or multiple processors or computers. The apparatus can include special purpose logic circuitry, e.g., an FPGA (field programmable gate array) or an ASIC (application specific integrated circuit) or RISC (reduced instruction set circuit). The apparatus can also include, in addition to hardware, code that creates an execution environment for the computer program in question, e.g., code that constitutes processor firmware, a protocol stack, an information base management system, an operating system, or a combination of one or more of them.

Data processing system 18 also includes memory 22 and a bus system 26, including, for example, a data bus and a motherboard, can be used to establish and to control data communication between the components of data processing system 18. Processing device 28 can include one or more microprocessors. Generally, processing device 28 can include an appropriate processor and/or logic that is capable of receiving and storing data, and of communicating over a network (not shown). Memory 22 can include a hard drive and a random access memory storage device, including, e.g., a dynamic random access memory, or other types of non-transitory machine-readable storage devices. Memory 22 stores data processing engine 30 that is executable by processing device 28. These computer programs may include a data engine (not shown) for implementing the operations and/or the techniques described herein. The data engine can be implemented in software running on a computer device, hardware or a combination of software and hardware.

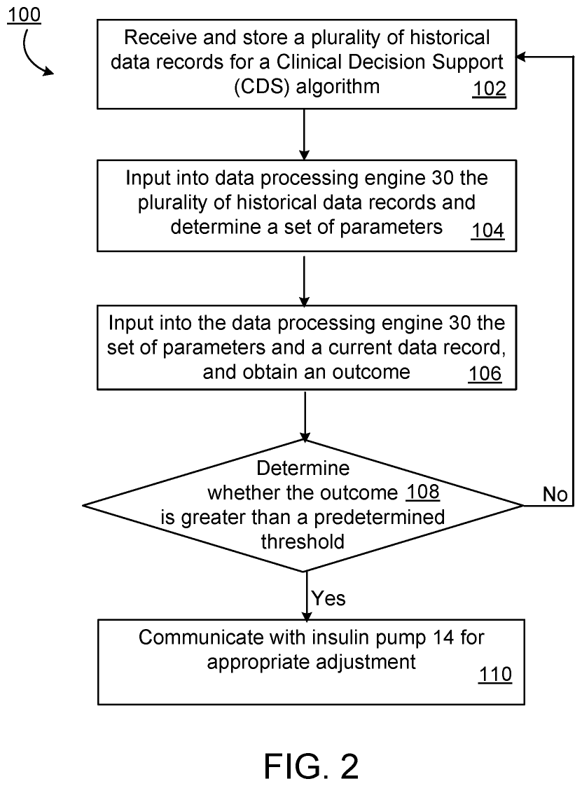

Referring to FIG. 2, data processing system 18 performs process 100 to output information indicative of an optimized value for an insulin pump parameter. In operation, data processing system 18 receives and stores data representing one or more predicting factors for a CDS algorithm (step 102). In some embodiments, the data are received at appropriate time intervals, e.g., 10 minutes, 20 minutes, 30 minutes, 1 hour, 2 hours, 1 day, 2 days etc. Data processing system 18 inputs into CDS data processing engine 30 data representing one or more predicting factors of a CDS algorithm (step 104). In some embodiments, the data can come from activity monitor 34, client device 32, blood glucose monitor 36, insulin pump 14, and/or client device 12. In some embodiments, the data are stored in data repository 20. Data processing system 18 then applies the CDS algorithm to the data (step 106), and determines the outcome. In some embodiments, data processing system 18 further determines whether the outcome is greater than a predetermined threshold (step 108). If the outcome is greater than a predetermined threshold, data processing engine 18 communicates with insulin pump 14 for appropriate adjustment (step 110), otherwise, data processing system 18 continues to receive and store data representing one or more predicting factors (step 102), e.g., data from activity monitor 34, client device 32, blood glucose monitor 36, insulin pump 14, and/or client device 12. In some embodiments, data processing system 18 outputs, by the one or more data processing devices 28, information indicative of the outcome of a CDS algorithm. The output may be transmitted to a display device, e.g., a CRT (cathode ray tube) or LCD (liquid crystal display) monitor, or transmitted to client device 12, client device 32, a third client device, insulin pump 14 through network 16, etc.

In some embodiments, data processing system 18 combines the data with CGM and pump data at regular intervals, allowing for an on-going analysis of trends in glucose metrics, e.g., fasting glucose, 2-hour postprandial glucose, and incidence of hypoglycemia.

Implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, i.e., one or more modules of computer program instructions encoded on a tangible program carrier for execution by, or to control the operation of, a processing device. Alternatively or in addition, the program instructions can be encoded on a propagated signal that is an artificially generated signal, e.g., a machine-generated electrical, optical, or electromagnetic signal that is generated to encode information for transmission to suitable receiver apparatus for execution by a processing device. A machine-readable medium can be a machine-readable storage device, a machine-readable storage substrate, a random or serial access memory device, or a combination of one or more of them.

In some embodiments, various methods and formulae are implemented in the form of computer program instructions and executed by processing device. Suitable programming languages for expressing the program instructions include, but are not limited to, C, C++, Java, Python, SQL, Perl, Tcl/Tk, JavaScript, ADA, OCaml, Haskell, Scala, and statistical analysis software, such as SAS, R, MATLAB, SPSS, CORExpress® statistical analysis software and Stata etc. Various aspects of the methods may be written in different computing languages from one another, and the various aspects are caused to communicate with one another by appropriate system-level-tools available on a given system.

The processes and logic flows described in this specification can be performed by one or more programmable computers executing one or more computer programs to perform functions by operating on input information and generating output. The processes and logic flows can also be performed by, and apparatus can also be implemented as, special purpose logic circuitry, e.g., an FPGA (field programmable gate array) or an ASIC (application specific integrated circuit) or RISC.

Computers suitable for the execution of a computer program include, by way of example, general or special purpose microprocessors or both, or any other kind of central processing unit. Generally, a central processing unit will receive instructions and information from a read only memory or a random access memory or both. The essential elements of a computer are a central processing unit for performing or executing instructions and one or more memory devices for storing instructions and information. Generally, a computer will also include, or be operatively coupled to receive information from or transfer information to, or both, one or more mass storage devices for storing information, e.g., magnetic, magneto optical disks, or optical disks. However, a computer need not have such devices. Moreover, a computer can be embedded in another device, e.g., a mobile telephone, a smartphone or a tablet, a touchscreen device or surface, a personal digital assistant (PDA), a mobile audio or video player, a game console, a Global Positioning System (GPS) receiver, or a portable storage device (e.g., a universal serial bus (USB) flash drive), to name just a few.

Computer readable media suitable for storing computer program instructions and information include various forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and (Blue Ray) DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back end component, e.g., as an information server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital information communication, e.g., a communication network. Examples of communication networks include a local area network (“LAN”) and a wide area network (“WAN”), e.g., the Internet.

The computing systems can include clients and servers. A client and server are generally remote from each other and typically interact through a communication network. The relationship of client and server arises by virtue of computer programs running on the respective computers and having a client-server relationship to each other. In some embodiments, the server can be in the cloud via cloud computing services.

While this specification includes many specific implementation details, these should not be construed as limitations on the scope of any of what may be claimed, but rather as descriptions of features that may be specific to particular implementations. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Particular implementations of the subject matter have been described. Other implementations are within the scope of the following claims. For example, the actions recited in the claims can be performed in a different order and still achieve desirable results. In one embodiment, the processes depicted in the accompanying figures do not necessarily require the particular order shown, or sequential order, to achieve desirable results. In some implementations, multitasking and parallel processing may be advantageous.

As described above, the CDS system can be configured to apply various CDS algorithms, e.g., Multivariate Statistical Model (MSM) for Predicting Therapy Adjustment (MSM-TA), Multi-Input-Multi-Output (MIMO) Adaptive Proportional Integral Derivative (APID) control algorithm (MIMO-APID), and optimal bolus estimation (OPT-BE) algorithm.

### Multivariate Statistical Model (MSM) for Predicting Therapy Adjustment (MSM-TA)

A limiting factor in improving the glucose control achieved by individuals with diabetes is the underlying day-to-day variability. Intermittently high fasting glucose levels cannot be corrected by adjusting insulin without placing subjects at risk for hypoglycemia on days where their fasting glucose is within an accepted euglycemic range. Likewise low nighttime glucose values cannot be corrected adjusting insulin doses without creating hyperglycemia on nights when the glucose is in target range. A completely analogous argument holds for meal insulin dosing. If a given bolus estimator is configured with parameters that provide good control for some meals, but not other meals, the parameters cannot be adjusted to bring the poorly controlled meals into target range without compromising the meals that are well controlled.

The present disclosure provides methods of determining an appropriate insulin dose at different time periods, for example, determining whether higher or lower insulin doses for a particular night and determining insulin bolus dose for a meal. In some embodiments, the described methods utilize the data available at the time the dosing adjustment needs to be effected, for example, before going to sleep, before a meal, after a meal, etc. The present disclosure also provides methods of determining an appropriate insulin bolus. The described methods identify which meals require adjusted dosing using the data available at the time the dose is calculated (in this case, just prior to the meal being consumed).

MSM's can be described as follows:

Outcomeᵢ=α₀+α₁Predictor₁+α₂Predictor₃+ . . . α_{N}Predictor_{N}+εᵢ Eq. 1

The key to realizing the benefit of these models is choosing an appropriate outcome and identifying appropriate predictors (or predicting factors). In Eq. 1, some exemplary outcomes include, but are not limited to, nighttime nadir glucose (NNG), morning fasting glucose (MFG), 2 and 5-hour postprandial glucose (PPG_{2HR} and PPG_{5HR}) and 5 hour nadir postprandial glucose (NPP_{5HR}). Numerous relevant predictors (or predicting factors) can be used in the MSM, e.g., daytime activity, meal fat content, and blood glucose level. Each outcome is described as having an underlying expected (mean) value (α₀), statistically significant predicting factors (Predictor_{1 . . . N}) with their corresponding coefficients (α₁, α₂, α₃, α₄ . . . ), together with an associated error, or variability about the mean, characterized by εᵢ. For example, the outcome variable NNG may have a mean value of 150 mg/dL (α₀) with normally distributed errors about the mean of 50 mg/dL (standard deviation of εᵢ). This would imply that ˜2.15% of values would be below 50 mg/dL and 2.15% above 250 mg/dL. If the underlying cause of the variability can be identified, e.g., if daytime activity predicts NNG (α₁ significantly different from zero; p<0.05), a recommendation can be effected to reduce or increase nighttime insulin use on the nights following high or low activity. In some embodiments, if fat content in the food predicts blood glucose level, a recommendation can be made to adjust the insulin dose for a meal in response to a meal with high fat content. In some embodiments, recommendations can be made to either a health care provider or patient, then the health care provider or the patient can take appropriate actions, and data processing system 18 can communicate with insulin pump 14 to effect the required adjustment.

In some embodiments, the parameters of MSM-TA algorithm can be identified by data records of a group of subjects. As such, each data record would refer to an individual subject and any one effect (e.g., α₁) would be identified by studying an appropriate number of subjects (appropriate being defined by power calculations).

In some embodiments, the MSM-TA algorithm is applied individually to each patient. In the implementation used in effecting CDS, each data record refers to an individual night or meal. The appropriate number of nights or meals needed to determine the effect in question is statistically significant can be set by performing a power calculation.

In some embodiments, the predictors (or predicting factors) are identified by the CDS algorithms. In some embodiments, the MSM-TA algorithm is configured to allow automatic adjustment to account for physiological change in a person (e.g., the significance of a predictor (or predicting factor) and the coefficients of a predictor (or predicting factor) can evolve over time). For example, activity may not predict NNG in a very young or very old subject, but may become statistically significant during puberty or other life changes. To account for this kind of change, the MSM-TA algorithm is configured to use either a fixed window of data (e.g., prior 3, 2, or 1 month, or 3, 2, or 1 week) or effect the solution with a “forgetting factor” (e.g., data 3 months old is given ½ the weight of that just obtained). Use of a “forgetting factor” allows equation 1 to be easily identified using a recursive form of the least-squares identification routine.

### Multi-Input-Multi-Output (MIMO) Adaptive Proportional Integral Derivative (APID) Control Algorithm (MIMO-APID)

The recommendation to increase or decrease an insulin dose for a specific meal or for a night can be provided to the patient or patients' caregiver. The exact amount and timing is determined by the MIMO-APID algorithm. The CDS algorithm is termed MIMO as multiple output values (e.g., glucose level at 3, 5 and 7 am, or 7, 9 and 12 pm) may depend on multiple inputs (e.g., basal rates from 12 am to 3 am, 3 am to 5 am, and 5 am to 7 am, or basal rates from 5 pm to 7 pm, 7 to 9 pm, and 9 to midnight plus the carbohydrate to insulin ratio used at dinner time). In some embodiments, changes in therapy settings are effected slowly over time using adaptive Proportional Integral Derivative (PID) control algorithms. The adaptive PID algorithm is implemented in an interacting form in which the P (proportional) and I (integral) terms are first calculated using an incremental form; i.e., incremental adjustments made in response to glucose above or below target (integral) and the rate of change of glucose (derivative). For example, the basal rate between midnight and 1 am (BASAL₀₁) on the most recent data available (BASAL₀₋₁^{N}) would be updated based on errors in the glucose values affected that day and their rate-of-change:

$${BASAL}_{0 - 1}^{N} = {BASAL}_{0 - 1}^{N - 1} + \frac{{k}_{1} [ {G}_{1 am}^{N - 1} - target ]}{{T}_{I}} + \frac{{k}_{2} [ {G}_{2 am}^{N - 1} - target ]}{{T}_{I}} + … + \\ \frac{{k}_{q} [ {G}_{q}^{N - 1} - target ]}{{T}_{I}} + {k}_{1} [ dGd {t}_{1 am}^{N - 1} ] + {k}_{2} [ dGd {t}_{2 am}^{N - 1} ] + … + {k}_{q} [ {dGdt}_{q}^{N - 1} ] \qquad Eq . 2$$

dGdt is a derivative. It is the actual rate of change—the actual number can be obtained from continuous glucose monitoring records. [G−target]/T is the implicit desired rate of change which changes as G goes to Target (at Target the desired rate is zero). In some embodiments, the basal rate is determined by glucose value that are observed 1-6 hours after the time period of interest in the previous day (e.g., rate used from 12:00 am to 1:00 AM is determined, in part, by glucose values observed at 2:00 AM, 3 AM, 4 AM etc).

Consider a simplified version of Eq. 2 which includes only the first proportional term and first derivative term:

$$B A S A {L}_{0 - 1}^{N} = B A S A {L}_{0 - 1}^{N - 1} + \frac{{k}_{1} [ {G}_{1 am}^{N - 1} - target ]}{{T}_{I}} + {k}_{1} [ d G d {t}_{1 am}^{N - 1} ]$$

If the glucose is above target—say 30 mg/dl high—and T_{I} and k₁ are set to 30 minutes and 0.1 U/h per mg/dl per min respectively. If dGdt is zero the basal rate will increase by 0.1 [30]/30, or 0.1 U/h. If glucose is falling at 1 mg/dl/min there will be no change in basal rate, and dGt in increasing by 1 mg/dL per min the basal rate will increase by 0.2 U/h. The fact that there is no change in basal when glucose is 30 mg/dl high and falling at 1 mg/dl per min implicitly means that the person that choose T_{I} wants the glucose to be falling at the rate [G−target]/T_{I}. Thus, the algorithm—a type of proportional integral control—is configured so that choosing T_{I} sets a desired rate of change.

Generally, q is chosen to allow glucose values at future time point to effect changes in basal rates ending at a previous time point. This is done to account for the delays observed in subcutaneously delivered insulin (i.e., the pharmacokinetic/pharmacodynamic or PK/PD delays).

The values for Ki are chosen, in part, based on the how comfortable the caregiver is in making large versus frequent adjustments and in part based on the PK/PD profile of the insulin used. The final values for BASAL achieved by the algorithm do not change with the choice of k−k determines how fast the algorithm converges. For example, if the current BASAL rate ending 1 AM is 0.5 U/h and the necessary BASAL rate is 1.0 U/h, choosing values of k that are small may result in 5 recommended changes of 0.1 U/h whereas larger values might result in the same increase (0.5 U/h) occurring over two changes with each change equal to 0.25 U/h. However, while 2 changes may be preferable to 5 changes (fewer decisions needing to made by the physician) there is an added risk that one of the changes will “overshoot” the necessary amount, creating a potentially unsafe condition and/or resulting in a third change where the rate is lowered. In some embodiments, the values of k can be made to adapt to the patients underlying insulin sensitivity such that individuals with high daily insulin requirements are managed with high values of k, and those with low insulin use are managed with lower values.

The rate of change of glucose level (G) is not based on the sample interval N (days)—i.e. not based on 3 am glucose value today minus the 3 am glucose value yesterday divided by 24 which is an indicator of how fast the algorithm is converging—but rather the rate of change at the time of the sample; i.e., the rate of change of glucose at 3 am on the current day. In some embodiments, this number is often available from the continuous glucose monitor (e.g., blood glucose monitor 36). The value of T_{I} is based on an implicit desired rate of glucose change, for example,

$$desired rate = \frac{[ G - target ]}{{T}_{I}} \qquad Eq . 3$$

Eq. 3 shows that the desired rate of change (desired rate) decreases as the blood glucose level (G) approaches the target value (target) and is set by the integration time T_{I} (e.g., 30 minutes, 45 minutes, 60 minutes). In some embodiments, the value of T_{I} for treating subjects with low or high glucose levels accompanied by symptom can be different from the value for treating subjects without symptoms. Generally, basal rates do not change day-by-day. In some embodiments, the changes only occur once a threshold difference is achieved, e.g.,

Σₙ₌₁^{q}|BASAL₀₋ₜ_{ₙ}^{N}−BASAL₀₋ₜ_{ₙ}^{in use}≥threshold Eq. 4

Eq. 4 shows that a change is made for BASAL rate when the difference between the basal rate in use and the current suggested basal rate is greater than a preset threshold. The threshold per se can be related to the patient's insulin sensitivity factor; for example, if the ISF is 1 Unit of insulin drops glucose 30 mg/dL might be set at ⅓ of a unit, as an expected change in glucose of less than 10 mg/dL might be considered clinically insignificant. (It is also anticipated that different Basal profiles will be set depending on the significance of different predicting factors as determined in Eq. 1. For example, if daytime activity or meal fat content is determined to predict nighttime nadir glucose, then separate basal rates would be determined for days following high fat, or high activity days.

In some embodiments, basal rates may be updated based on a single event; in particular, the symptomatic hypo- or hyperglycemia may effect an immediate change whereas the same glucose value unaccompanied by symptoms would contribute to a possible change following the rules established in equations 2-4.

In Eq. 3, the desired rate of change (desired rate) decreases as the blood glucose level (G) approaches the target value (target) and is set by the integration time T_{I} (e.g., 30 minutes, 45 minutes, 60 minutes).

In some embodiments, when the threshold difference is achieved, and an incremental adjustment is required the adjustment may be less than the threshold (e.g., threshold, threshold/2 or threshold/3).

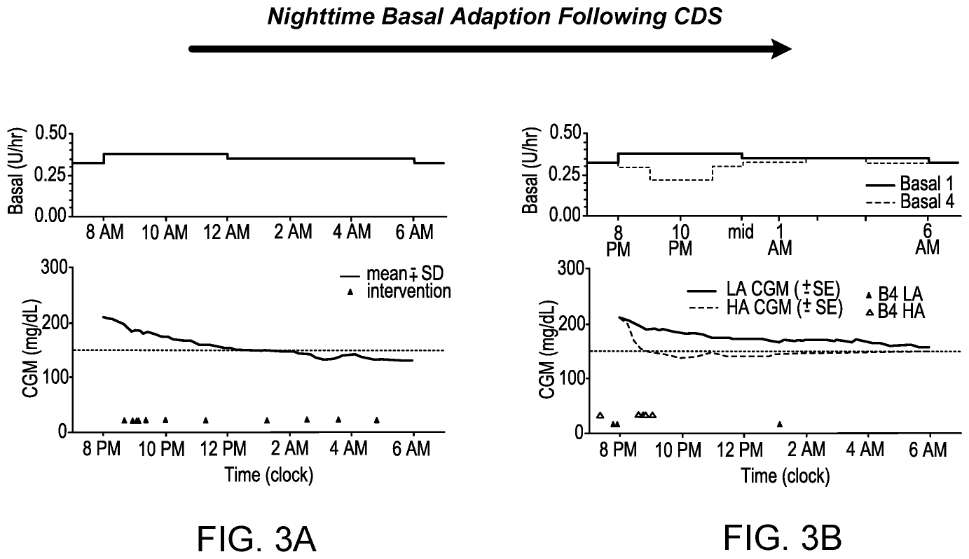

FIG. 3a shows nighttime basal rates for a 7 year old boy (top panel) and corresponding CGM glucose (lower panel). Closed triangles along the bottom of the graph indicate the use of supplemental carbohydrate to prevent or correct hypoglycemia.

FIG. 3b shows nighttime basal rate adaption for the same subject as determined by the MIMO-APID algorithms described herein. Activity (Low activity, LA; high activity, HA) is measured by a FitBit® step count with data collected at a defined time (e.g., 8 PM) allowing that day's activity to be used to effect changes in the nighttime basal profile (e.g., 8 PM to 6 AM profile) prior to patient going to bed. As activity is identified as a predictor of nighttime nadir glucose, nighttime basal rates have been adjusted to account for different levels of activities. Fewer supplemental carbohydrates are required to correct hypoglycemia. In some embodiments, morning activity may be treated differently from afternoon activity.

Predictors are identified using multivariate statistical analysis with predefined outcomes (e.g., nadir nighttime glucose or morning 6 AM glucose, use of supplemental carbohydrates or insulin correction boluses). Significance is assessed using statistical methodology (e.g., testing whether regression line relating daytime step count to nighttime nadir glucose is statistically different from 0 by F-test; use of chi2 analysis on the use of supplemental carbohydrate or insulin correction boluses separated by activity). Wherever possible, statistical analysis is performed using recursive relationships (e.g., recursive least squares to update slope and intercept of regression lines).

Many predictors can be used. For example, exercise decreases nighttime basal, fat and protein increase meal insulin requirement. Other potential predictors include, but are not limited to, psychological factors, menstrual cycle (for women), etc.

### Use of a Metabolic Model to Guide Therapy Adjustment

It is often difficult to simultaneously adjust meal insulin doses together with basal rates per se. This is particularly true as many meal boluses are given as extended or dual wave boluses. The underlying idea is to give a calculated DOSE (U of insulin) in two parts—one part being an immediate bolus (percentage of dose range 0 to 100% and the second part as an infusion (U/hour) over a specified duration (e.g., typically 0.5 to 6 hours). To improve optimization under these conditions, the described methods introduce a metabolic model characterized by a limited set of identifiable parameters (e.g., parameters describing the insulin PK/PD curve, parameters characterizing the effect of insulin to lower blood glucose, the effect of glucose per se to increase glucose uptake into cells and decrease endogenous glucose production, parameters describing gastric emptying, etc.).

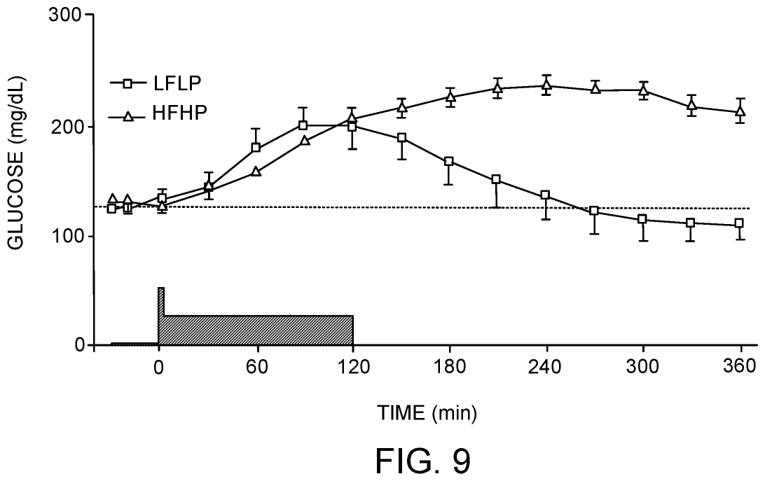

In some embodiments, model parameters are then identified for problem meals and the model is used to calculate optimal bolus pattern (optimal dose, percent given as a bolus, and duration for the remaining insulin to be given). For example, in studies performed in individuals consuming a pizza meal with and without cheese it is often observed that the addition of cheese (addition of fat and protein) results in prolonged postprandial hyperglycemia. FIG. 9 shows results of comparing a pizza without cheese (labeled low fat low protein or LFLP) and with cheese (labeled high fat high protein or HFHP) in 10 individuals with type 1 diabetes. Both meals had the identical carbohydrate amount (50 grams) differing only in fat (4 v 44 grams) and protein (9 versus 36 grams). In both meals subjects initially gave insulin following their standard CIR ratio with 50% given as an immediate bolus and 50% given over a two hours DURATION (shown in Figure as grey shaded region). That the LFLP meal returns to target (dashed line) within approximately 3 hours suggest that the insulin DOSE (U) was appropriate for a LFLP meal; that the HFHP meal did not return to Target within 6 hours indicates an alternate bolus—either amount or pattern—is needed.

While it is clear that a different bolus is, on average, needed to cover the HFHP meal no methodology currently exists to calculate how the bolus should be adjusted. We propose to calculate the optimal DOSE, SPLIT (% given as an immediate bolus) and duration using a model. An example, taken from one of the subjects studied in FIG. 9, serves to illustrate the individual steps.

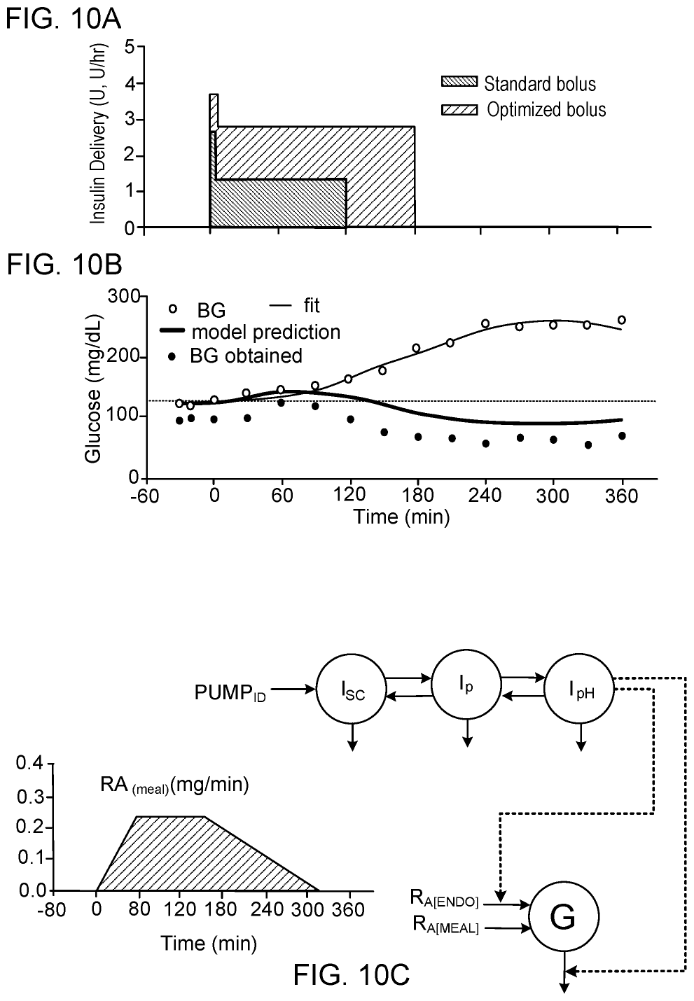

The first step in obtaining an optimal model predicted bolus (MPB) for a meal with an inappropriate glucose profile is fit to the BG values obtained to a metabolic model that predicts the glucose response based on how many grams of carbohydrate were consumed and how much insulin was given (FIGS. 10a and 10b). We choose a low order model—i.e., a model with the minimal number of equations and parameters needed to fit the data. The model is shown in FIG. 10c, and is comprised of a 3-compartment insulin PK/PD model together with a one-compartment glucose model. In the 3 compartment PK/PD model, insulin is delivered into the space immediately below the skin (subcutaneous space with concentration denoted I_{SC}). This forms the first compartment. From there, insulin is absorbed into the vascular or plasma compartment (second compartment, concentration denoted I_{P}) from which is it distributed into the interstitial fluid (ISF) surrounding insulin sensitive tissue (third compartment, concentration denoted I_{ISF}). A one compartment model is used to describe glucose concentration (concentration denoted, G). This compartment is assumed to be comprised of the plasma (fluid that blood cells reside in) and interstitial fluid in tissue beds that rapidly equilibrate with plasma (primarily gut and splanchnic bed). Insulin is assumed to act by increasing glucose uptake from the compartment (down arrow leaving the space) and decreasing the rate of endogenous glucose appearance into the compartment (glucose released by liver and kidneys). Insulin is assumed to act in proportion to the insulin levels in the ISF compartment (effect on liver/kidneys and peripheral glucose uptake shown with blue dash lines). Negative values are assumed to correspond to conditions where the liver and kidneys take up more glucose than they release (sometimes referred to as net hepatic glucose balance). The rate of appearance of glucose derived from a meal is denoted R_{A[MEAL]} and is described is described by initial rise in glucose appearance lasting Tᵣᵢₛₑ minutes, followed by a constant rate of appearance lasting Tc minutes, followed by a linear decrease in appearance lasting T_{decrease} minutes. Total area under the curve is equal to the grams of carbohydrate consumed in the meal. The 3 meal parameters (Tᵣᵢₛₑ, T_{constant}, and T_{decrease}), along with 3 time constants describing the PK/PD model (τ₁, τ₂, τ₃), a glucose distribution space parameter (V, indicating size of compartment G in dL), a fractional glucose clearance at basal insulin parameter (p₁) and the combined effect of insulin to increase peripheral glucose uptake and decrease endogenous glucose production (insulin sensitivity parameter, S_{I}) result in nine identifiable parameters. Model equations are:

$$\frac{{dI}_{S C}}{d t} = \frac{1}{{τ}_{1}} Δ ID - \frac{1}{{τ}_{1}} {I}_{S C}$$

$$\frac{{dI}_{p}}{d t} = \frac{1}{{τ}_{2}} {I}_{S C} - \frac{1}{{τ}_{2}} {I}_{p}$$

$$\frac{{dI}_{ISF}}{d t} = \frac{1}{{τ}_{3}} {I}_{P} - \frac{1}{{τ}_{3}} {I}_{ISF}$$

$$\frac{d G}{d t} = - [ {p}_{1} + {S}_{I} {I}_{ISF} ] G + \frac{1}{{V}_{G}} [ {V}_{G} {p}_{1} {G}_{B} + {R}_{A [ M E A L ]} ]$$

$${R}_{A [ M E A L ]} = if {T}_{MEAL} \leq t < {T}_{r i s e} {SLOPE}_{1} [ t - {T}_{M E A L} ] if {T}_{r i s e} \leq t < {T}_{c o n s t a n t} {R}_{A [ MAX ]} if {T}_{c o n s t a n t} \leq t \leq {T}_{decrease} {SLOPE}_{2} [ t - {T}_{c o n s t a n t} ]$$

The parameters are identified using nonlinear least squares routines, which minimized the sum square error between the observed BG values (i.e., the values in the undesirable meal response) and the model predicted values (G in the above equation). In alternate embodiments CGM glucose can be replace BG measurements per se. Setting total area under the curve for RA_{[MEAL]} equal grams carbohydrate consumed in the meal reduces the number of parameters to be identified in the meal response to 3. For the example subject chosen, the optimized model fit is shown as the red line.

The second step involves using the model to predict what the glucose response would look like with a different insulin bolus; i.e., a different DOSE, SPLIT, or DURATION. While a trial and error approach can be used to obtain a more desirable response we propose to identify the optimal settings by minimizing a cost function. For the data shown (optimal model predicted bolus shown in FIG. 10a blue shaded region; predicted glucose response shown in FIG. 10b blue line) we chose a cost function that minimized the area below target for the first 120 minutes post meal, and the difference above target in the interval from 120 minutes to 360 minutes. That is, we defined the cost J as:

j=∫₀^{120 min}(Area below target)dt+∫₁₂₀³⁶⁰(Area above target)dt

We choose this cost function as we noted in some instances minimizing the total area different from target resulted in the meal response initially decreasing. Other cost functions are also possible. In particular, cost functions in which a high and low target are set:

J=∫₀^{120 min}(Area below target_{LOW})dt+∫₁₂₀³⁶⁰(Area below target_{LOW})dt+∫₁₂₀³⁶⁰(Area Above target_{HIGH})dt

Or where hypoglycemia is given greater weight than hyperglycemia

J=weight₁∫₀^{120 min}(Area below target_{LOW})dt+weight₁∫₁₂₀³⁶⁰(Area below target_{LOW})dt+weight₂∫₁₂₀³⁶⁰(Area Above target_{HIGH})dt

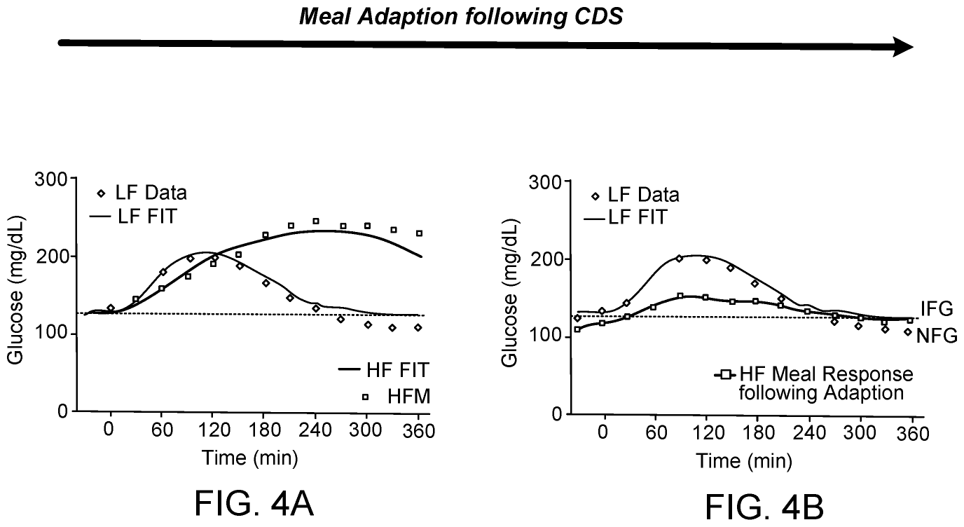

In addition to minimizing the cost function, the adaptation algorithm makes use of constraints. For the data shown, optimization was performed subject to the constraint that the total DOSE not increase by more than 75% on any one iteration. In some instances this constraint resulted in an unacceptable meal response on a subsequent visit. In these instances the procedures were repeated (fit meal, optimize with constraint new bolus DOSE not greater than previous bolus DOSE time 1.75). In some embodiments, the described methods make even small incremental adjustments (limit the increase between successive to 50%, 25% or 10%). This increases safety as it allows the algorithm to account for intraday variability. Average meal responses obtained with the procedure are shown FIG. 4.

For patients who do not use complex bolus patterns—e.g., patients using Multiple Daily Injection therapy—the meal bolus may be adapted following similar MIMO-adaptive-PID rules to those proposed for adapting basal. Here, the bolus is linked to post prandial peak, 2 hour and nadir glucose values (G_{PPP}, G_{2hrPP}, G_{NPP}). To this end, an optimal CIR would then be estimated for a meal consumed on that specific day (denoted CIR_{OPT}^{N} where OPT indicates optimal, N defines the specific meal and day). Over a period of time, the CIR can adapt according to:

CIR_{new}^{N+1}=CIR_{new}^{N}+k·(CIR_{OPT}^{N}−CIR_{in use}^{N}) Eq. 5

In Eq. 5, CIR^{N}_{OPT} is the optimal CIR as determined. CIR^{N}_{in use} is the CIR that is currently in use, k is a number less than 1 (vector of magnitude <1 in the case of a dual wave or bolus pattern defined by more than 1 parameter). Eq. 5 provides that the new CIR is only adjusted for a portion of the difference between CIR^{N}_{OPT} and CIR^{N}ᵢₙ use. Setting k to a small value (e.g., ⅕, ¼, ⅓, or ½) requires multiple meals with observed high or low glucose values. This provides robust adjustments accounting for model error and interday variability.

CIR^{N}_{OPT} is usually obtained by a optimizing a model. For example, CIR^{N}_{OPT} may be determined by “model independent” adaptive routines. We define a target incremental peak post prandial glucose value (TARGET_{PPP}) that goes up with increasing meal size:

TARGET_{PPP}=k_{desired}MEAL_{CHO} Eq. 6a

Typical value for k_{desired} would be 1; i.e., a 100 gram meal would increases glucose 100 mg/dL. We then link the CIR to difference between the observed and target desired peak postprandial glucose,

CIR^{N}=CIR^{N-1}−k₂(OBSERVED_{PPP}−TARGET_{PPP})

if |CIR_{new}^{N}−CIR_{in use}^{N}|≥CIR_{CHANGE threshold}

then CIR_{in use}^{N+1}=CIR_{in use}^{N}+CIR_{CHANGE threshold} Eq. 6b

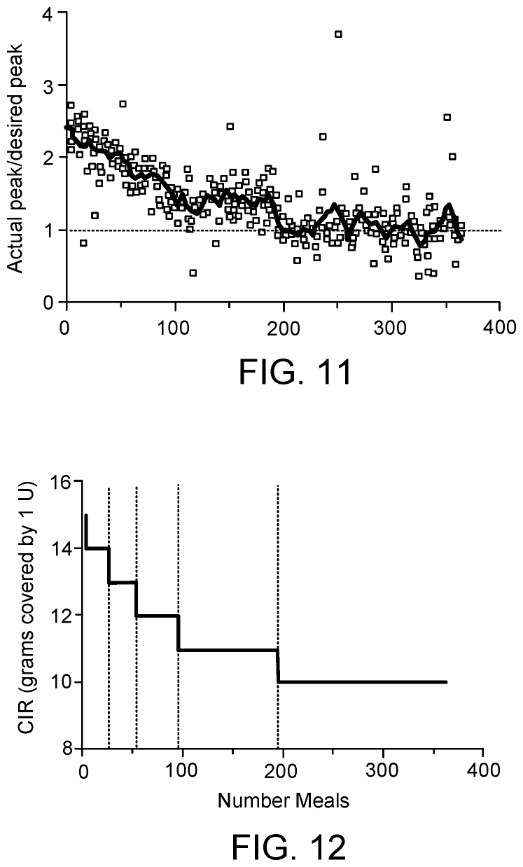

Equation effectively mimics how physicians “titrate dosing” for many drugs—including insulin. That is, physicians will often have a TARGET in mind, and if the target is not achieved they incrementally increase or decrease the DOSE (new DOSE=old DOSE plus incremental change). When doing this, care needs to be taken to not react to fast to any given observation, as there can be substantial day-to-day or meal-to-meal variability unrelated to the dose. Thus, repeat—or consistent—observations of a higher than TARGET_{PPP} are often required before deciding to increase the dose. In this formulation, the need for a consistent pattern is determined by parameter k₂. Setting the value small will protect against making spurious recommendations but slow the algorithms convergence. The ability to prevent spurious recommendations is shown in FIG. 11 using a simple Excel simulation. In the simulation we assume a virtual patient is consuming meals between 15 and 120 grams, with number of grams taken from a uniform random distribution. Initially, the meals result in an average peak post prandial glucose concentration of 2.5 times the number of grams (e.g., a 100 gram meal increases glucose 250 mg/dL) when using a CIR of 1 U covers 15 grams. For the simulation we consider this rise to have a random component that is normally distributed with mean zero and standard deviation 10 mg/dL; i.e., assume spurious noise with standard deviation 10 mg/dL. We set a desired peak at 1 times the amount of carbohydrate consumed and plot in the simulation the ratio of obtained peak and desired peak (a value of 1 indicating good control, values higher than 1 indicating postprandial hyperglycemia).

We set the CIR change threshold to 1, meaning we change the CIR “in use” whenever the integer portion of the CIR calculated by 6b decreases by 1. We begin the simulation with CIR set at 15 and incremental peak postprandial glucose 2.5 times the grams of carbohydrate consumed. We also assume a CIR of 1 U covers 10 grams will lead to good control and that the decrease is peak postprandial glucose is linear with changes in CIR (this last assumption is not necessary as the algorithm will converge for both a linear and nonlinear system providing the algorithm is configured with an appropriate choice of k₂ and CIR_{CHANGE THRESHOLD} For the simulation we set k₂ to 0.000003 and set the CIR^{N-1} to 15, meaning any initial hyperglycemia will decrease the CIR to less than 15 prompting the first change (FIG. 11). Results illustrate that the algorithm converges to the correct CIR over a couple of months, with 4 intermediate incremental changes (FIG. 12) and no spurious, or undesired, increases. Different choices for k₂ and CIR_{[CHANGE THRESHOLD]} will result in faster or slower convergence with more or less changes, but will not affect the final value achieved.

In some embodiments the algorithm may be effected with additional rules that treat symptomatic hypo or hyperglycemia differently than biochemical hypo or hyperglycemia, with, for example, as single event of symptomatic hyper or hypoglycemia being sufficient to recommend increasing or decreasing the CIR (a common symptom of hyperglycemia is elevates ketones; a common symptom of hypoglycemia includes lethargy).

|CIR_{new}^{N}−CIR_{in use}^{N}|≥CIR_{CHANGE threshold} Eq. 6c

Eq. 6b shows that a change is made for CIR when the difference between CIR^{N}_{new} and CIR^{N}_{in use} is greater than a predetermined threshold CIR_{change threshold}. The new CIR would be recommended only once it differs from the value in use by a predetermined threshold (similar to Eq. 4) as shown in Eq. 6c.

### Optimal Bolus Estimation (OPT-BE)

Dietary fat and protein can increase postprandial glucose concentrations in patients with type 1 diabetes. In 2015, the American Diabetes Association recommended that people with type 1 diabetes who have mastered carbohydrate counting should receive education on the impact of protein and fat on glucose control. Dietary fat can cause significant hyperglycemia in the late postprandial period (>3 h) due to free fatty acid (FFA)-induced peripheral insulin resistance and increased hepatic glucose output. There is a need for more definitive experimental data to guide clinical practice recommendations for patients with type 1 diabetes on how to adjust prandial insulin doses for higher fat and higher protein meals.

The present disclosure relates to an Optimal Bolus Estimator (OPT-BE). The CDS system can be used to adapt the configuration of the OPT-BE (CDS will adapt any bolus estimator). The OPT-BE differs from other bolus estimators effectively supporting two unmet needs. First, in some embodiments, it considers how the different nutritional components of a meal interact when estimating insulin dosing patterns whereas existing bolus calculators rely almost exclusively on carbohydrate content when meal calculating insulin doses. Second, in some embodiments, OPT-BE takes into consideration previously unavailable information on the rate of change of the glucose concentration and the rate of change of insulin-on-board. Many bolus estimators typically rely only on glucose concentration and assume insulin-on-board to be decreasing at all points other than when a new correction bolus is input.

Many Bolus Estimators that exist today suffer from similar problems: the estimators do not effectively incorporate meal nutrient components other than carbohydrate, they do not include the glucose rate-of-change information available from continuous glucose monitors, and they do not include the information available regarding directional changes in insulin-on-board. They were also designed to work exclusively with pump-therapy, and the estimators assume the pump basal rates are correct at the time the bolus is calculated.

In contrast, in some embodiments, the OPT-BE algorithm is designed to be equally effective for pump and MDI patients. In some embodiments, the OPT-BE algorithm includes glucose rate-of-change information. In some embodiments, the OPT-BE algorithm includes directional IOB information.

### Overview

The Bolus Estimator described herein determines correction boluses based on an insulin sensitivity factor (ISF), and protect against so-called insulin stacking through the use of an insulin-on-board calculation. Although differences exist on how IOB is calculated in different pumps, the basic construct is in the form:

$$correction bolus = \frac{[ BG - target ]}{ISF} - IOB \qquad Eq . 7$$

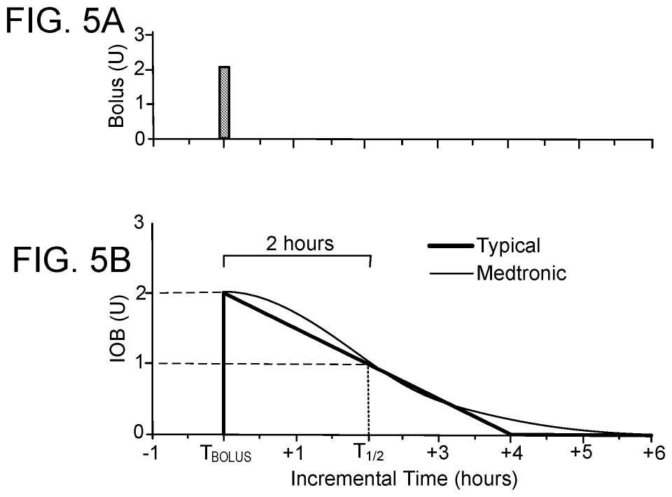

In Eq. 7, BG is the blood glucose level, target is the target blood glucose level, ISF is Insulin Sensitivity Factor, and IOB is insulin on board. IOB depends on the amount and timing of the last correction bolus (typically, doses calculated to cover carbohydrate are not included in IOB). Generally, the IOB is characterized by a ½-time (time for the insulin effect to dissipate 50%) as shown in FIGS. 5a-5b. The calculation typically assumes a linear approximation (Blue line) but can in some cases be calculated from an insulin PK/PD curve (Red line). A general description for PK/PD curves can be found, e.g., in Insulin aspart (B28 asp-insulin): a fast-acting analog of human insulin: absorption kinetics and action profile compared with regular human insulin in healthy nondiabetic subjects. Mudaliar S R, Lindberg F A, Joyce M, Beerdsen P, Strange P, Lin A, Henry R R. Diabetes Care. 1999 September; 22(9):1501-6.

In FIGS. 5a-5b, a subject has given themselves a 2 U bolus at time TBOLUS (FIG. 5a) and the T_{1/2} has been set at 2 hours in both approaches (FIG. 5b). IOB time is one of the parameters set in virtually all insulin pumps; however, each pump uses slightly different curves and slightly different definitions. A detailed description regarding IOB time can be found, e.g., in Bolus calculator: a review of four “smart” insulin pumps. Zisser H1, Robinson L, Bevier W, Dassau E, Ellingsen C, Doyle F J, Jovanovic L. Diabetes Technol Ther. 2008 December; 10(6):441-4.

The bolus could originate, for example, in a subject who has a target glucose of 120 mg/dL, an ISF of 1 U decreases glucose 30 mg/dL, no IOB and who measures their glucose and finds it to 180 mg/dL. Under this condition the correction bolus would be calculated as [180−120]/30−0=2 U. Note that if the patient has an IOB that equals 2 U, the recommended bolus would be [180−120]/30−2=0. This illustrates the ability IOB to limit any new bolus from being delivered until the insulin already given has had time to act (referred to as protection against over-stacking). Further note that the IOB sets an implicit expectation for a decrease in glucose. For this example, 2 U would be expected to decreases the glucose level by 60 mg/dL, with a drop of 30 mg/dL expected in the first 2 hours (IOB T_{1/2}). Thus, if the subject enters a BG value of 150 mg/dL 2 hours later, the bolus estimator calculates [150−120]/30−1=0 (i.e., recommend 0 U insulin). If glucose was above 150 mg/dL at this time a bolus would be recommended; however, if glucose were below 150 mg/dL no action would be taken.

In the examples described above, the bolus estimator calculations can provide protection against over stacking, but they do not take into account the glucose rate-of-change information from CGM. The methods described herein address these issues by OPT-BE. In some embodiments, OPT-BE can be applied to an insulin therapy pump. In other embodiments, OPT-BE can be applied to MDI patients, and the patients receive multiple daily insulin injection based on the bolus calculated by OPT-BE.

### Rate of Change

In some embodiments, the rate of change of glucose level is introduced into OPT-BE. In some embodiments, the OPT-BE incorporates the concept of desired rate of change in Eq. 3. In one example, a subject has a target glucose of 120 mg/dL, an ISF of 1 U decreases glucose 30 mg/dL, no IOB, and a measured BG of 150 mg/dL. In this example, a standard BE would recommend a correction bolus of 1 U as shown in Eq. 8:

$$correction bolus = \frac{[ BG - target ]}{ISF} - IOB = \frac{[ 150 - 120 ]}{30} - 0 = 1 U \qquad Eq . 8$$

A detailed description regarding how to calculate a standard BE can be found, e.g. in Bolus calculator: a review of four “smart” insulin pumps. Zisser H1, Robinson L, Bevier W, Dassau E, Ellingsen C, Doyle F J, Jovanovic L. Diabetes Technol Ther. 2008 December; 10(6):441-4.

However, individuals should not be given this bolus if the glucose level is already rapidly falling

$$( \frac{dG}{dt} < - 2 \frac{mg}{dl} per min ) ,$$

or should be given a larger bolus if their glucose level is rapidly rising

$$( \frac{dG}{dt} > 2 \frac{mg}{dl} per min ) .$$

In some embodiments, blood glucose monitor 36 routinely reports these rises as 1, 2, or 3 arrows (changing 1-2 mg/dL per min, 2-3 mg/dL per min, and changing more than 3 mg/dL per min). In the case where glucose level is falling at 2 mg/dL per min, a measured glucose value 30 mg/dL above target will reasonably be expected to resolve itself within 15 minutes, or even raise concerns regarding possible hypoglycemia. In some cases, glucose that is stable at 150 mg/dL is viewed as being too slow (give bolus) and glucose falling at 3 or mg/dL too fast (perhaps suspend pump), while values of 0.5 to 1 mg/dL per min should be seen as reasonable (no correction needed).

The OPT-BE incorporates the concept of desired rate of change in Eq. 3. The result is Eq. 9.

$$correction bolus = \frac{{k}_{1} [ BG - target ]}{{T}_{I}} + \frac{{k}_{1} d G}{d t} - IOB \qquad Eq . 9$$

In Eq. 9, k₁ may be set in proportion to the individual's insulin sensitivity (S_{I}); i.e., someone with low S_{I} would be provided with a large value for k₁; someone with high sensitivity would be provided with a low value. In some embodiments, the value may be adapted to provide a rate of convergence consistent with what the physician would do in normal practice; i.e., if the physician frequently overrides the algorithm with a bigger or smaller change the algorithm would adapt to mimic what the physician would do.

BG is the blood glucose level, target is the target blood glucose level, the value of T_{I} is based on an implicit desired rate of change of glucose, dG/dt is the actual rate of change of the blood glucose level. Where setting T_{I}=30 minutes results in a desired rate of fall 1 mg/dL per minute when glucose is 30 mg/dL above target. A more conservative value of T_{I}=60 minutes would result in a desired rate of change of 0.5 mg/dL per min. Table 1 shows the estimated bolus dose as determined by OPT-BE assuming K₁=2, IOB=0 and T_{I}=60 minutes. Table 2 shows the estimated bolus dose as determined by OPT-BE assuming K₁=1, IOB=0 and T_{I}=60 minutes

TABLE 1
K₁ = 2; T₁ = 60 min

| | | dG/dt | | | | |
|---|---|---|---|---|---|---|
| | | −1 | −0.5 | 0 | 0.5 | 1 |
| BG | 210 | 1 | 2 | 3 | 4 | 5 |
| | 180 | 0 | 1 | 2 | 3 | 4 |
| | 150 | — | 0 | 1 | 2 | 3 |
| | 120 | — | — | 0 | 1 | 2 |

TABLE 2
K₁ = 1; T₁ = 60 min

| | | dG/dt | | | | |
|---|---|---|---|---|---|---|
| | | −1 | −0.5 | 0 | 0.5 | 1 |
| BG | 210 | 0.5 | 1 | 1.5 | 2 | 2.5 |
| | 180 | 0 | 0.5 | 1 | 1.5 | 2 |
| | 150 | — | 0 | 0.5 | 1 | 1.5 |
| | 120 | — | — | 0 | 0.5 | 1 |

Tables 1 and 2 show the following:
Irrespective of gain (K₁=1 or 2) glucose above target but falling at the desired rate leads to a correction bolus recommendation of zero (no bolus)
Glucose values falling faster than target can be used to recommend temporary suspension of basal rates
For K₁=2 (Table 1) the column corresponding to dG/dt=0 behaves identically to existing bolus estimators configured with ISF of 1 U decreases glucose 30 mg/dL.
Glucose at target but increasing can lead to a preemptive recommendation to give a bolus.

### IOB Tracking

The IOB calculations can protect against insulin over stacking. However, a more in-depth examination of how the calculations are performed shows the calculation can be improved.

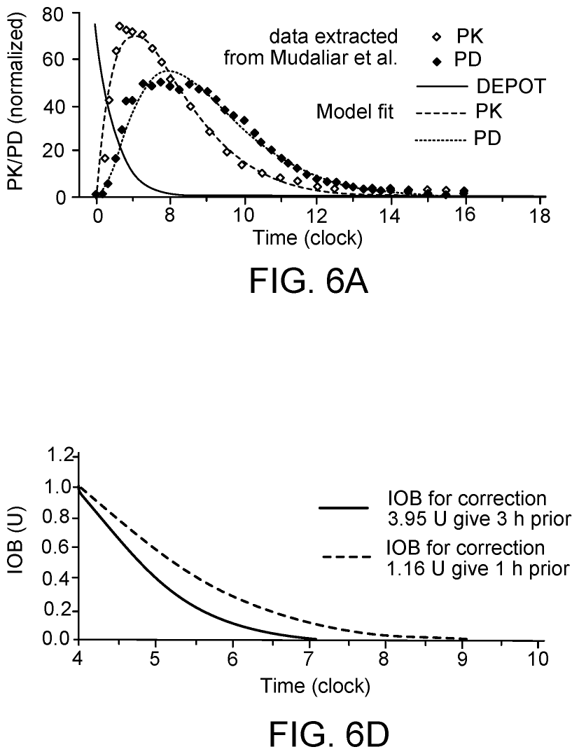

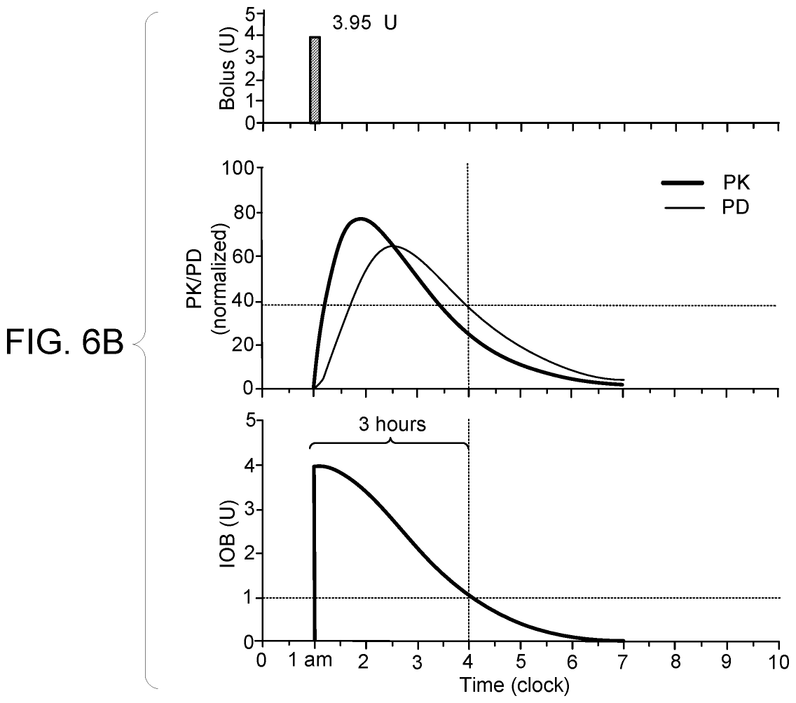

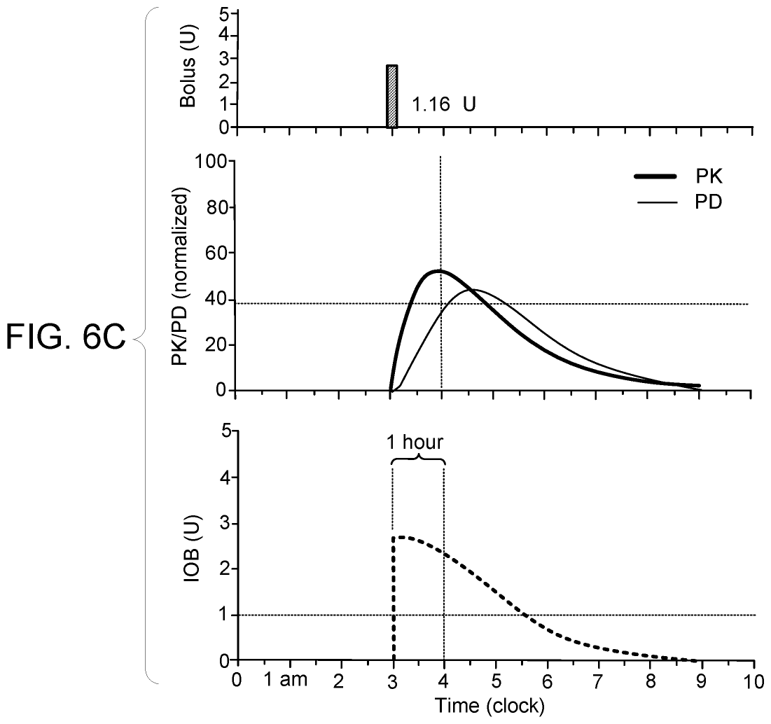

For example, in FIG. 3, the shape of the IOB curve is derived from the known PK/PD insulin response. Generally, the PK/PD curve can be fit to a 3-compartment as shown FIG. 6a. The IOB curve is then calculated for a 1 U bolus as:

$$IOB ( t ) = \frac{{\int }_{0}^{∞} P {D}_{MODEL} ( t ) d t - {\int }_{0}^{t} P {D}_{MODEL} ( t ) d t}{{\int }_{0}^{∞} P {D}_{MODEL} ( t ) d t} \qquad Eq . 10$$

In Eq. 10, PD_{MODEL}(t) is the function for PD curve. IOB(t) is the insulin on board at time point (t) with the total IOB at time point 0 is adjusted for 1 U bolus. IOB(t) can be subsequently scaled up or down for boluses of different magnitudes.

The problem with the approach—which we address with our revised OPT-BE—is that any given IOB number can be obtained in two different ways. For example, with an IOB half-life of 2 hours, 2 U given 2 hours ago results in an IOB of 1 U. The same 1 U IOB can be obtained from a 1 U bolus just given. A more complicated example—shown in FIG. 6—shows IOB for 3.95 U bolus given 3 hours in the past and IOB for 1.16 Units given 1 hour in the past. The values are chosen to highlight:
In both instances IOB at 4 am is equal to 1 U.
In the first instance (FIG. 6b) the PK curve is below the PD curve and both curves are decreasing
In the second instance (FIG. 6c) the PK curve is above the PD curve and at its maximal level; the PD curve is below the PK curve
IOB is identical at 4 am but remains higher for values after 4 am for 1.16 U given 1 hour in the past.
   Of these 4 points, only the 4^{th} point is consistent and this point is only true of the Medtronic IOB curve. The Medtronic IOB curve was derived from the PK/PD response described in Mudaliar S R, Lindberg F A, Joyce M, Beerdsen P, Strange P, Lin A, Henry R R. Diabetes Care. 1999 September; 22(9):1501-6. In short, the curve was obtained as:

$$IOB ( t ) = \frac{[ {\int }_{0}^{360} {G}_{INF} dt - {\int }_{0}^{t} {G}_{INF} dt ]}{{\int }_{0}^{360} {G}_{INF} dt}$$

In this curve, the shape is monotonically decreasing at all time-points except for the instance that a correction bolus is given to the subject. Points 1-3 are inconsistent and can create erratic behavior where in one instance the correction bolus yield may yield the desired effect (bring glucose from a high value to target) and in another case generate hypoglycemia or fail to bring glucose to target in the desired time frame. Generally, if the effect is increasing at the time of the correction bolus the bolus can be decreased. The problem addressed by the OPT-BE is the loss of directional information in IOB calculation—which results in an expected waning of the effect (IOB is always decreasing when a correction bolus is calculated).

To address this problem, the OPT-BE retains information as to the relative magnitude of each of each PK/PD component:

IOB(t)=a₀·I_{SC}+a₁·I_{P}+a₂·I_{EFF} Eq. 11

Where I_{SC} is the concentration of insulin at the subcutaneous injection site, I_{P} is the concentration of insulin in plasma, and I_{EFF} is the effect profile, which is delayed relative to changes in plasma insulin.

Eq. 11 retains information relating to the relative magnitudes of the insulin PK and PD curves and takes into account whether they are increasing or decreasing. The OPT-BE can prevent insulin stacking as the subcutaneous depot always increases by the bolus amount at the time the bolus is given, thereby preventing a second bolus being given before insulin has had time to act. Parameters a₀, a₁, and a₂ can be optimized using metabolic model simulation by the methods described, e.g., in Loutseiko M, Voskanyan, G, Keenan, D B, Steil, G M: Closed-Loop Insulin Delivery Utilizing Pole Placement to Compensate for Delays in Subcutaneous Insulin Delivery. Journal of Diabetes Science and Technology 5:9 (2011).

### Meal Bolus Estimation

BE typically treats carbohydrate as the only nutritional component of importance. Mainly, the BE proceeds in the form:

$$BE = CHO \text{/} CIR + [ \frac{[ BG - target ]}{ISF} - IOB ] \qquad Eq . 12$$

Eq. 12 incorporates Eq. 7. It further adjusts BE based on the grams of carbohydrate to be consumed (CHO). CIR is a Carbohydrate to Insulin ratio (expressed as the number of grams covered by 1 U of insulin). Generally, IOB is not subtracted from the calculation for new insulin to cover added carbohydrates, but low blood sugar corrections are (e.g., if BG is 60 below target with an ISF of 1 U decreases 30 mg/dL, and the subject is to consume 30 grams carbohydrate and has a CIR 1 U covers 10 grams the recommended bolus would be 1 U not 3; precise details may differ among different BE).

However, nutrients other than carbohydrate can influence insulin requirements (Bell K J, Smart C E, Steil G M, Brand-Miller J C, King B, Wolpert H A: Impact of Fat, Protein, and Glycemic Index on Postprandial Glucose Control in Type 1 Diabetes: Implications for Intensive Diabetes Management in the Continuous Glucose Monitoring Era. Diabetes Care 38:1008-1015, 2015). The described methods herein shift the paradigm from carb counting per se, to a meal-centric bolus estimation (MCBE). Using MCBE, subjects will tag specific meals that they frequently eat. In practice, many subjects have a set of meals they frequently consume. In some embodiments, the described methods identify the optimal bolus for these meals using a two-step process. In step 1, the meal response is obtained using the subject's standard, but not necessarily optimal, bolus estimate. The response is then fit to a low-order identifiable (LOI) metabolic model (MM) and the LOI-MM used to calculate an optimal BE for the meal consumed that day (denoted BE_{OPT}^{N} where OPT indicates optimal, N defines the specific meal and day).

The next time the subject consumes the same meal a new recommendation is provided (BE_{REC}^{N+1}). The new recommendation is not the optimal value but rather a bolus that takes a small step in the direction of the optimal bolus. Specifically,

BE_{REC}^{N+1}=BE_{REC}^{N}+k·(BE_{OPT}^{N}−BE_{REC}^{N}) Eq. 13

In Eq. 13, BE^{N}_{OPT} is the optimal BE as determined by Eq. 12, BE^{N}_{REC} is the recommended BE. A new recommended BE (BE^{N+1}_{REC}) is determined by adjusting BE^{N}_{REC} for a portion (k) of difference between BE^{N}_{OPT} and BE^{N}_{REC}. k is a number less than 1 (e.g., 0.2, 0.5; vector of magnitude <1 in the case of a dual wave or bolus pattern defined by more than 1 parameter). Setting k to a small value requires multiple meal with observed high or low glucose values. This provides robust adjustments accounting for model error and interday variability. The new BE would take effect only once the difference from a current recommendation reaches a predefined threshold, similar to the strategy outlined for CDS changes in BASAL and CIR:

|BE_{REC}^{N}−BE_{in use}^{N}|≥BE_{CHANGE threshold} Eq. 14

Eq. 14 shows that a change is made for BE^{N}_{REC} when the difference between BE^{N}_{REC} and BE^{N}_{in use} is greater than a predetermined threshold (BE_{change threshold}). In some embodiments, the optimization process is done in data processing system 18.

Once a significant number of tagged meals are optimized, the nutritional content of these meals can be applied to a multivariate statistical model similar to Eq. 1, but with the inclusion of so-called interaction terms. Interaction terms allow for the possibility that the effect of carbohydrate content per se may vary at different levels of fat. Both fat and carbohydrates would be included as so-called main effects. In principle, the BE can be generalized to a function as shown in Eq. 15:

BE=α₁CHO+α₂FAT+α₃PROTEIN+α₁₂CHO*FAT+α₁₃CHO*PROTEIN+α₂₃PROTEIN*FAT Eq. 15

In Eq. 15, the outcome is BE. The predicting factors include CHO, FAT, PROTEIN, and the interaction terms CHO*FAT, CHO*PROTEIN and PROTEIN*FAT. α₁, α₂, α₃, α₁₂, α₁₃, α₂₃ are the associated coefficients. In some embodiments, the coefficients are determined by the analysis of variance (ANOVA). In some embodiments, Eq. 15 will not include main effects or interactions that cannot be shown statistically significant by ANOVA. In some embodiments, other appropriate predictors can be added in Eq. 15 (e.g., alcohol or coffee).

### Bolus Acceleration

It has long been recognized that one of the limitations to subcutaneous (SC) insulin delivery is added delay associated with SC-insulin absorption. Progress has been made in this area with the introduction of monomeric or rapid acting insulin insulins. As well, research continues with companies looking to add compounds that may make the absorption even faster (hyaluronidase), add heat or mechanically stimulate the site (vibrations) to the site, or inject the insulin intradermally rather than subcutaneously. In some embodiments, the described methods utilize model predicted insulin feedback (MPIF) per se.

MPIF is obtained using a subset of the model equations used in the MPB procedures (model shown FIG. 10c); specifically, the 3-equations describing insulin concentrations at the insulin delivery site (I_{SC}), plasma (I_{P}), and ISF surrounding insulin sensitive tissue (I_{ISF}):

$$\frac{{dI}_{SC}}{d t} = \frac{1}{{τ}_{1}} {BOLUS}_{ID} - \frac{1}{{τ}_{1}} {I}_{S C}$$

$$\frac{{dI}_{p}}{d t} = \frac{1}{{τ}_{2}} {I}_{SC} - \frac{1}{{τ}_{2}} {I}_{p}$$

$$\frac{{dI}_{ISF}}{d t} = \frac{1}{{τ}_{3}} {I}_{P} - \frac{1}{{τ}_{3}} {I}_{ISF}$$

Where the first equation has been modified to reflect the observation that pump insulin deliver (U/hr) to typically broken up into multiple small boluses given at discrete time points (e.g. 1 U/h may be delivered as a series of 0.05 U boluses given every 3 minutes; 0.05 U being is the minimum stroke volume many pumps are able to deliver). If the time each individual bolus is given is known, the equations can be implemented in a more computationally efficient form using z-transforms. Tables of Z-transforms can be found in in numerous text-books, e.g., Franklin G F and Powell J D, Digital Control of Dynamic Systems Addison-Wesley Publishing, 1980.

### EXAMPLES

The invention is further described in the following examples, which do not limit the scope of the invention described in the claims.

### Example 1: Optimizing Mealtime Insulin Dosing

Experiments were performed to demonstrate the importance of considering meal composition in determining mealtime insulin doses.

Research Design and Methods

Subjects:

Ten adults with type 1 diabetes using continuous subcutaneous insulin infusion (CSII) and continuous glucose monitoring (CGM) were recruited through the Joslin Clinic. To be eligible, subjects had to be aged 18-75 years, have had type 1 diabetes for >3 years, been on insulin pump therapy for >6 months, and have an HbA1c<8.5%. Those with celiac disease, dietary restrictions, medications that affect insulin sensitivity, gastric motility, digestion or absorption disorders or who were pregnant, breastfeeding or planning to become pregnant were excluded. The study was approved by the Joslin Diabetes Center Institutional Review Board.

Study Protocol:

In the 3 weeks prior to commencing the study, subjects attended clinic appointments to review and optimize their basal rates, insulin sensitivity factor (ISF) and carbohydrate-to-insulin ratio (CIR). The day prior to each admission, subjects had a new CGM sensor and insulin pump infusion catheter inserted. They were then instructed to consume a low fat dinner meal that night, avoid alcohol and vigorous physical activity, and not consume additional food after 10 PM other than supplemental carbohydrate to correct hypoglycemia.

Subjects presented to the Clinical Research Center (CRC) at the Joslin between 8:00-9:00 AM (10-11 h fast). On admission, an intravenous catheter for frequent blood sampling was inserted, the fasting blood glucose concentration determined, and the pump changed to an Animas Ping pump (West Chester, Pa.). If the glucose concentration was above the target range (80-130 mg/dL), a correction insulin dose was administered and the test session delayed for 2.5 h. If the baseline level was below target, the subject was treated and the test session commenced after 2.5 h.

On the first two visits, subjects consumed the LFLP and HFHP meal in random order. The prandial bolus was calculated using their individualized CIR and was delivered as a dual wave bolus, with a 50/50% over 2 hours at the beginning of the meal. Since the carbohydrate content of the two meals was identical, the insulin doses were also identical. On up to 4 subsequent visits, subjects repeated the HFHP meal with an insulin dose estimated using an adaptive model predictive bolus (MPB) algorithm. Visits were repeated until target postprandial glycemic control was achieved (see Adaptive MPB algorithm below). If hypoglycemia occurred during a test session, the subject was treated with glucose tablets until blood glucose levels returned to target, the event and treatment noted, and the session continued. At the conclusion of the session, the study insulin pump was disconnected and the patient resumed their usual care blood glucose management. Venous blood samples were taken at −30, −20 and 0 minutes prior to the test meal and then every 30 minutes for the following 6 hours. Glucose levels were analyzed using a YSI 2300 glucose analyzer (YSI Life Sciences, Yellow Springs, Ohio).

Diet Intervention:

Meals were prepared the morning of the test session in the CRC kitchen. The meals consisted of a commercially available pizza base marinara sauce (LFLP meal) or the same pizza base and sauce with added cheese (HFHP meal). Nutrition information for the test meals is reported in Table 3. The two meals where matched for carbohydrate (50 g) but varied in protein, fat and calories. The LFLP meal contained 273 calories, 9 g protein and 4 g fat whereas the HFHP meal contained 764 calories, 36 g protein and 44 g fat. The pizza base had a glycemic index (GI) of 52 (unpublished data).

Adaptive MPB Algorithm:

Insulin dose and delivery pattern was adjusted using a MPB. The MPB algorithm was applied in two steps. In the first step, metabolic model parameters were identified from the HFHP meal. The metabolic model Shown FIG. 10c, allowed 9 parameters to be identified: 3 insulin PK/PD rate constants, volume of the glucose compartment, a glucose effectiveness rate constant, insulin sensitivity normalized to insulin clearance, and 3 parameters characterizing the initial rise, maximum rate, and fall in glucose appearance following the meal. Optimal parameter estimates were obtained using a nonlinear generalized reduced gradient algorithm programed in Microsoft Excel (Office 2013). We have previously used a similar model to characterize the effect of meal fat content on insulin requirements [10] and characterize intraday changes in metabolism [11, 12].

In the second step, a model derived optimal insulin DOSE (U), SPLIT (percent given as bolus), and DURATION (time in minutes to give remaining DOSE), was obtained by minimizing the model predicted glucose area below target during the first 120 minutes following the meal plus the model predicted area above target from 120 to 360 minutes. The same nonlinear generalized reduced gradient algorithm described above was used but with an added constraint limiting the maximum increase in DOSE between study visits to 1.75 times the current dose (maximum DOSE for visit 3 equal 1.75 usual care DOSE). If the maximum DOSE proved to be insufficient, or was otherwise not able achieve target glucose values, subjects returned to the CRC on a later date. Target glucose values were considered to be acceptable (no further visits required) when the following 4 criteria were achieved:
1) Not more than 10 mg/dL decrease from baseline (BL) during the first 120 minutes of the meal
2) Peak postprandial glucose ≤BL plus 80 mg/dL
3) Two-hour postprandial glucose ≤BL plus 40 mg/dL
4) Six-hour postprandial glucose within 20 mg/dL of BL

statistical analysis: average glucose profile are shown as mean±standard error (SE). Incremental area under the curve (iAUC) was calculated using trapezoidal integration with BL calculated as the average glucose in the 30 minutes preceding the meal. Changes in insulin DOSE and iAUC were assessed by repeated measures analysis of variance with p<0.05 considered significant. Multiple comparisons were corrected using Dunnett's procedure with the LFLP meal taken as comparison (HFHP meal and MPB meal compared to LFLP meal if the overall ANOVA was significant). Patient demographics are reported as mean and standard deviation (SD). Linear regression was used to assess significance of demographic characteristics on predicting insulin dose adjustments (i.e., fat and protein sensitivity). Statistical testing was done using Graphpad Prism V 6.04.

Results

Patient Characteristics.

Ten patients (9 male, 1 female) were recruited for the study from the Joslin Diabetes Clinic. The mean age was 60.4±11.3 years, Body Mass Index (BMI) was 25.8±3.5 kg/m² (SD), HbA1c was 7.1±0.8% (54±7 mmol/mol). Subjects had been diagnosed with type 1 diabetes for an average of 46.1±15.4 years and been using CSII for an average of 13.7±5.1 years.

LFLP Meal Vs. HFHP Meal.

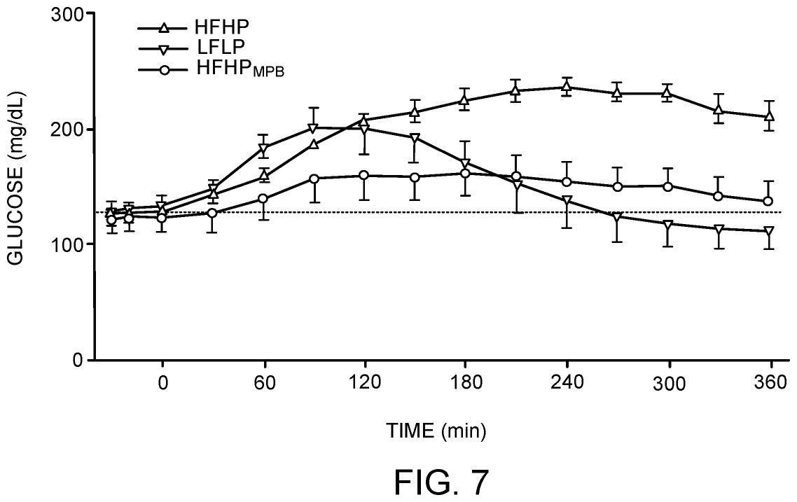

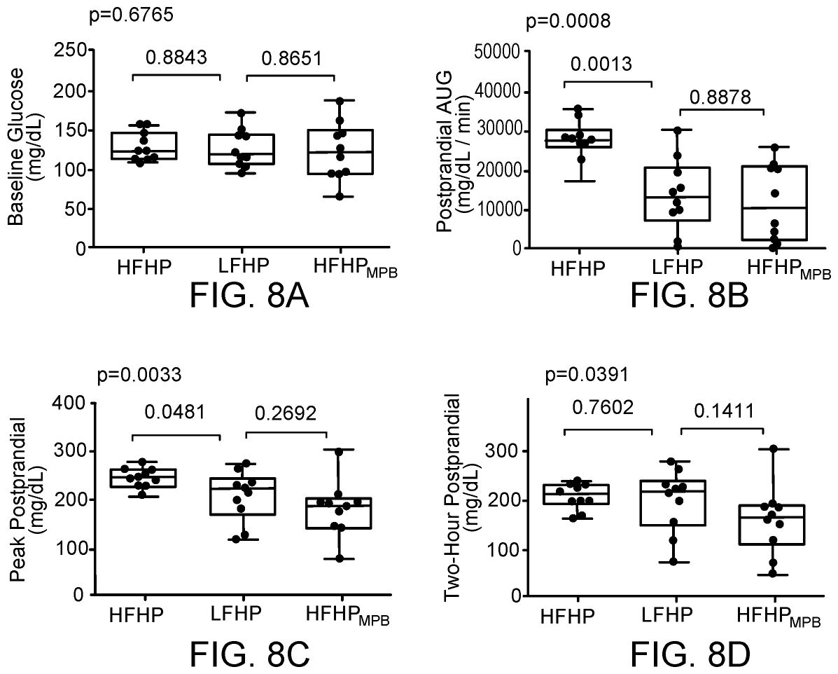

The mean insulin dose delivered for the LFLP and HFHP meals using the subject's individual CIR was 4.7±0.6 units. There were no significant differences in the fasting blood glucose level between the two study days (FIG. 7 and FIG. 8; Table 3; 127±8 mg/dL vs. 129±5 mg/dL, p=0.702). However, with the same insulin dose, the HFHP meal more than doubled the iAUC (Table 4; 27092±1709 vs. 13320±2960 mg/dL-min; p=0.0013). The mean incremental blood glucose concentration was significantly increased following the HFHP meal compared with the LFLP meal (+73±4 mg/dL vs.+23±11 mg/dL, p=0.001), with significant differences from 180 minutes onwards. At the conclusion of the 6 h study, the mean glucose level was 100 mg/dL higher following the HFHP meal compared with the LFLP meal. The mean incremental peak blood glucose concentration was 36 mg/dL higher following the HFHP meal compared with the LFLP meal (+118±7 mg/dL vs.+82±13 mg/dL, p=0.014) and was delayed by 150 minutes for the HFHP meal (255±21 minutes vs. 105±14 minutes, p<0.001).

Three subjects had a hypoglycemic episode requiring treatment with the LFHP meal whereas no subjects experienced hypoglycemia with the HFHP meal. Hypoglycemia occurred in the late postprandial period, with all 3 events occurring between 210-300 minutes.

TABLE 3
Nutritional composition of test foods

| | | Weight | Energy | CHO | Glycemic | Fat | Protein |
|---|---|---|---|---|---|---|---|
| Meal | Ingredient | (g) | (kCal) | (g) | Index (%) | (g) | (g) |
| Low Fat, Low | Pizza Base | 93 | 249 | 46 | 52 | 3 | 8 |
| Protein | Marinara | 42 | 24 | 4 | — | 1 | 1 |
| (LFLP) | Sauce | | | | | | |
| | TOTAL | 135 | 273 | 50 | | 4 | 9 |
| High Fat, | Pizza Base | 93 | 249 | 46 | 52 | 3 | 8 |
| High Protein | Marinara | 42 | 24 | 4 | — | 1 | 1 |
| (HFHP) | Sauce | | | | | | |
| | Cheese | 125 | 491 | 0 | — | 40 | 27 |
| | TOTAL | 260 | 764 | 50 | | 44 | 36 |

| | | | | |
|---|---|---|---|---|
| Difference | +491 | — | +40 | +27 |

TABLE 4
Mealtime insulin dosing and 6 hour postprandial blood
glucose profiles in 10 adults with type 1 diabetes

| | | | HFHP- |
|---|---|---|---|
| | | | Optimized |
| | LFLP | HFHP | Dose |
| Mean Insulin Dose | 4.7 | 4.7 | 7.9 |
| (units) | | | |
| Mean Insulin | 50/50 | 50/50 | 30/70 |
| Combination | | | |
| Wave Split (%/%) | | | |
| Mean Insulin | 120 | 120 | 144 |
| Combination | | | |
| Wave Duration | | | |
| (minutes) | | | |
| iAUC (mg/dL · min) | 13320 ± 2960 | 27092 ± 1709 | 11712 ± 3172 |
| Mean Incremental | +23 ± 11 | +73 ± 4 | +24 ± 11 |
| BGL (mg/dL) | | | |
| Mean Incremental | +82 ± 13 | +118 ± 7 | +61 ± 13 |
| Peak BGL (mg/dL) | | | |
| Time to Mean | 105 ± 14 | 255 ± 21 | 207 ± 33 |
| Incremental | | | |
| Peak BGL (minutes) | | | |
| Frequency of | 3 | 0 | 0 |
| hypoglycemia | | | |
| requiring treatment | | | |

LFLP = Low fat, low protein meal;
HFHP = High fat, high protein meal

Optimized Insulin Dose.

On average, it took 1.5 sessions to optimize the glycemic response, with 60% of participants achieving an optimize response on the first attempt. Need for repeat visits were primarily due to the safety constraint imposed on the MPB which limited the insulin dose increase to a maximum 75% increase per session. The mean insulin dose required to optimize glucose control was a 65±10% increase over the individualized CIR. There was considerable inter-individual variability, with insulin dose increases ranging from 17-124%. The smallest increase occurred in the subject with the lowest BMI and the largest increase in the subject with the highest BMI, with the regression slope BMI vs percent increase different from zero (p=0.0115). The optimal bolus delivery pattern was a dual wave bolus, with on average a 30/70% split over 2.4 h; optimal delivery patterns ranging from 10/90% to 50/50% split, with the extended bolus lasting from 2-3 h).

For the same HFHP meal, the optimized insulin dose significantly improved the iAUC compared with usual care dose (decreased iAUC from from 27092±1709 to 11712±3172) with the average iAUC not different from that observed with the LFLP meal (13320±2960 mg/dL min). The mean blood glucose concentration was significantly lower using the optimized insulin dose compared with the CIR (+24±11 mg/dL vs.+73±4 mg/dL; p=0.001), with significant differences from 120 minutes onwards. The mean incremental peak blood glucose concentration was 57 mg/dL lower using the optimized bolus (+61±13 mg/dL vs.+118±7 mg/dL, p=0.001) and occurred 48 minutes earlier compared with the CIR (207±33 minutes vs. 255±21 minutes, p=0.223). No subjects had a hypoglycemic episode requiring treatment using the optimized insulin dose.

This is the first study to use a model-based approach to derive an optimized insulin dose for open loop control of higher fat and protein foods by patients with type 1 diabetes. The addition of 40 g of dietary fat and 27 g of protein to 50 g of carbohydrate caused significant postprandial hyperglycemia 3-6 h when the insulin was calculated based on the CIR and carbohydrate content alone. To achieve target postprandial blood glucose control, the mean insulin dose needed to be increased by 65±10% over the individualized CIR and delivered as a dual wave with a 30/70% split over 2.4 h.

Applying the findings from our study, we recommend that for high fat meals (>40 g of fat) as a starting point patients should consider increasing the total insulin dose (calculated based on carb content and CIR) by 25-30%, and using a dual wave bolus with 30-50% upfront and the remainder delivered over 2-2.5 h. If review of glycemic profiles from the meal shows late (>3 h) increase in glucose concentrations, with subsequent similar meals the insulin dose delivered in the extended bolus should be increased. Review of early postprandial profile will provide insight about whether the amount of insulin delivered upfront in the combo bolus needs to be adjusted. For patient on injection therapy the combo bolus can be mimicked by taking a preprandial injection of regular+/−rapid-acting analog insulin or, alternatively, an injection of analog insulin preprandially followed by an additional injection 60-90 min later. There is experimental evidence from studies in non-diabetic individuals indicates that aerobic activity attenuates FFA-induced insulin resistance [18]. Although the effect of aerobic activity on fat sensitivity in individuals with diabetes is not known, we believe that until there is definitive data on this matter patients with diabetes should be counseled that it is prudent to be cautious when taking additional insulin to cover higher fat meals consumed following a bout of exercise.

To our knowledge this is the first study to use a model predictive control method to obtain an optimal magnitude and pattern for an open-loop meal bolus. To date, the use of models to optimize insulin dosing has primarily been limited to closed-loop artificial pancreas systems [24]. In this study, we replaced the Hovorka model with a piecewise linear approximation characterized by a linear increase (T_{RISE}) to maximal value (R_{aMAX}), and linear decrease (T_{FALL}) as shown FIG. 10c R_{A[MEAL]}. Use of the piecewise approximation adds two parameters to the meal rate-of-glucose appearance formulation (characterized as rise, maximal, and fall time rather than a single time-of-maximal appearance; T_{MAX}) but allowed more freedom in extending the time period over which the meal glucose was assumed to be absorbed from the gut. More sophisticated metabolic models exist that could potentially be used, but require the addition of glucose tracers [25], or add to the number of parameters needing to be identified without substantially improving the model fit [26]. Also, in our implementation we used the complete post-prandial response obtained on one day to predict the bolus that should be used on a subsequent day rather than glucose profile up to specific time point to predict future time points, as is done in traditional MPC control. To this end, our approach is similar to a “run-to-run” adaptive strategy [27] but with the difference being that a model is used to optimize the delivery pattern. Finally, our MPB optimization criteria was substantially weighted towards preventing any early postprandial hypoglycemia in that we minimized the decrease in glucose during the initial 120 minutes of the meal. We also included a safety constraint limiting the incremental increase in DOSE between repeat meals to be less than 1.75 the current DOSE.

While the model used for optimization the meal bolus [10-12] was chosen for its simplicity and ease of identification, it will likely require an app, or a modification to an existing pump bolus estimator, before it can be widely adapted; i.e. before it can be used to directly impact clinical practice. To this end, we believe the “carb-counting”paradigm will need to be replaced with a more “meal centric” paradigm—perhaps targeting specific meals the patient routinely eats. Further validation of the MPB algorithm in which a more complex variety of meals are optimized is also warranted. It should be noted that pizza may be easier to optimize as an identical mix of CHO, fat, and protein is generally consumed with subsequent meals. This is in contrast to mixed meals where the order in which different constituents are eaten can affect the glucose and insulin responses [28].

In summary, this example: 1) demonstrates that to optimize postprandial glucose control in type 1 diabetes some mealtime insulin doses need to be based on the meal composition rather than carbohydrate content only, and 2) provides the foundation for the development of new insulin dosing algorithms to cover high fat, high protein meals. The MPB approaches used here can produce optimal meal profiles in just one or two iterations and provides a means to systematically assess and clinically validate the required bolus pattern. Digital health tools will open up the opportunity to develop cloud-based systems that could remotely evaluate postprandial glucose profiles and apply this MPB approach to provide customized insulin dosing recommendations for specific meals to patients with diabetes.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### LIST OF REFERENCES

1. Bell K, Smart C, Steil G M, Brand-Miller J, King B R, Wolpert H. Impact of fat, protein and glycemic index on postprandial glucose control in type 1 diabetes: Implications for intensive diabetes management in the continuous glucose monitoring era. Diabetes Care. 2015; 38:1008-15.
2. American Diabetes Association. Standards of Medical Care in Diabetes 2015: Approaches to Glycemic Treatment. Diabetes Care. 2015; 38(Suppl. 1):S41-8.
3. Wolpert H A, Smith S A, Atakov-Castillo A, Steil G M. Dietary Fat Acutely Increases Glucose Concentrations and Insulin Requirements in Patients With Type 1 Diabetes: Implications for carbohydrate-based bolus dose calculation and intensive diabetes management. Diabetes Care. 2013; 36(4):810-6.
4. Wolever T M S, Mullan Y M. Sugars and fat have different effects on postprandial glucose responses in normal and type 1 diabetic subjects. Nutr Metab Cardiovasc Dis. 2011; 21:719-25.
5. Smart C E M, Lopez P E, Evans M, Jones T W, O'Connell S M, Davis E A, et al. Both Dietary Protein and Fat Increase Postprandial Glucose Excursions in Children With Type 1 Diabetes and the Effect Is Additive. Diabetes Care. 2013; 36:3897-902.
6. Sherwin R S, Kramer K J, Tobin J D, Insel P A, Liljenquist J E, Berman M, et al. A model of the kinetics of insulin in man. J Clin Invest. 1974 May; 53(5):1481-92.
7. Bergman R N, Finegood D T, Ader M. Assessment of insulin sensitivity in vivo. Endocr Rev. 1985 Winter; 6(1):45-86.
8. Caumo A, Bergman R N, Cobelli C. Insulin sensitivity from meal tolerance tests in normal subjects: a minimal model index. J Clin Endocrinol Metab. 2000 November; 85(11):4396-402.
9. Wilinska M E, Chassin L J, Acerini C L, Allen J M, Dunger D B, Hovorka R. Simulation environment to evaluate closed-loop insulin delivery systems in type 1 diabetes. J Diabetes Sci Technol. 2010 January; 4(1):132-44.
10. Laxminarayan S, Reifman J, Edwards S S, Wolpert H, Steil G M. Bolus Estimation-Rethinking the Effect of Meal Fat Content. Diabetes Technol Ther. 2015 December; 17(12):860-6.
11. Kanderian S S, Weinzimer S, Voskanyan G, Steil G M. Identification of intraday metabolic profiles during closed-loop glucose control in individuals with type 1 diabetes. J Diabetes Sci Technol. 2009 September; 3(5):1047-57.
12. Kanderian S S, Weinzimer S A, Steil G M. The identifiable virtual patient model: comparison of simulation and clinical closed-loop study results. J Diabetes Sci Technol. 2012; 6(2):371-9.
13. Garcla-Lopez J M, Gonzalez-Rodriguez M, Pazos-Couselo M, Gude F, Prieto-Tenreiro A, Casanueva F. Should the Amounts of Fat and Protein Be Taken into Consideration to Calculate the Lunch Prandial Insulin Bolus? Results from a Randomized Crossover Trial. Diabetes Technol Ther. 2013; 15(2):166-71.
14. Lodefalk M, Aman J, Bang P. Effects of fat supplementation on glycaemic response and gastric emptying in adolescents with Type 1 diabetes. Diabet Med. 2008; 25(9):1030-5.
15. De Palma A, Giani E, Iafusco D, Bosetti A, Macedoni M, Gazzarri A, et al. Lowering Postprandial Glycemia in Children with Type 1 Diabetes After Italian Pizza “Margherita” (TyBoDi2 Study). Diabetes Technol Ther. 2011; 13(4):483-7.
16. Chase H, Saib S, MacKenzi T, M M. H, S K. G. Postprandial glucose excursions following four methods of bolus insulin administration in subjects with type 1 diabetes. Diabet Med. 2002; 19:2146-51.
17. Jones S M, Quarry J L, Caldwell-McMillian M, Mauger D T, Gabbay R A. Optimal Insulin Pump Dosing and Postprandial Glycemia Following a Pizza Meal Using the Continuous Glucose Monitoring System. Diabetes Technol Ther. 2005; 7(2):233-40.
18. Schenk S, Horowitz J F. Acute exercise increases triglyceride synthesis in skeletal muscle and prevents fatty acid-induced insulin resistance. J Clin Invest. 2007; 117(6): 1690-8.
19. Pankowska E, Szypowska A, Lipka M, Szpotanska M, Blazik M, Groele L. Application of novel dual wave meal bolus and its impact of glycated hemoglobin A1C levels in children with type 1 diabetes. Pediatr Diabetes. 2009; 10(5):298-303.
20. Pankowska E, Blazik M, Groele L. Does the fat-protein meal increase postprandial glucose level in type 1 diabetes patients on insulin pump: the conclusion of a randomized study. Diabetes Technol Ther. 2012; 14(1): 16-22.
21. Kordonouri O, Hartmann R, Remus K, Blasig S, Sadeghian E, Danne T. Benefit of supplementary fat plus protein counting as compared with conventional carbohydrate counting for insulin bolus calculation in children with pump therapy. Pediatr Diabetes. 2012; 13:540-4.
22. Bao J, Gilbertson H R, Gray R, Munns D, Howard G, Petocz P, et al. Improving the Estimation of Mealtime Insulin Dose in Adults With Type 1 Diabetes: The Normal Insulin Demand for Dose Adjustment (NIDDA) study. Diabetes Care. 2011; 34(10):2146-51.
23. Bell K J, Gray R, Munns D, Petocz P, Howard G, Colagiuri S, et al. Estimating Insulin Demand for Protein-Containing Foods Using the Food Insulin Index. Eur J Clin Nutr. 2014; 68:1055-9.
24. Thabit H, Tauschmann M, Allen J M, Leelarathna L, Hartnell S, Wilinska M E, et al. Home Use of an Artificial Beta Cell in Type 1 Diabetes. N Engl J Med. 2015 Nov. 26; 373(22):2129-40.
25. Basu A, Basu R. Insulin:Carbohydrate Ratio-Part of the Story. Diabetes Technol Ther. 2015 December; 17(12):851-3.
26. Steil G M. Algorithms for a closed-loop artificial pancreas: the case for proportional-integral-derivative control. J Diabetes Sci Technol. 2013; 7(6): 1621-31.
27. Palerm C C, Zisser H, Bevier W C, Jovanovic L, Doyle F J, 3rd. Prandial insulin dosing using run-to-run control: application of clinical data and medical expertise to define a suitable performance metric. Diabetes Care. 2007 May; 30(5):1131-6.
28. Shukla A P, Iliescu R G, Thomas C E, Aronne L J. Food Order Has a Significant Impact on Postprandial Glucose and Insulin Levels. Diabetes Care. 2015 July; 38(7):e98-9.

1. A computer-implemented method of predicting a blood glucose level of a subject, the method comprising:
(1) receiving and storing a plurality of historical data records representing one or more predicting factors of the subject and a corresponding blood glucose level of the subject for a past period of time;
(2) inputting into a data processing engine the plurality of historical data records, and determining a set of parameters corresponding to the historical data records;
(3) inputting into the data processing engine the set of parameters and a current data record representing one or more predicting factors of the subject, thereby predicting a blood glucose level of the subject corresponding to the current data record; and
(4) outputting information indicative of the predicted blood glucose level corresponding to the current data record.

2. The method of claim 1, wherein the blood glucose level is nighttime nadir glucose (NNG), morning fasting glucose (MFG), 2-hour postprandial glucose (PPG2HR), 5-hour postprandial glucose (PPG5HR), or 5 hour nadir postprandial glucose (NPP5HR).

3. The method of claim 1, wherein the historical data records representing one or more predicting factors comprise a data record of a level of physical activity.

4. The method of claim 3, wherein the level of physical activity is measured by a continuous activity monitor.

5. The method of claim 1, wherein the historical data records representing one or more predicting factors comprise a data record of the fat content of a meal and/or the carbohydrate content of a meal.

6. The method of claim 1, wherein the historical data records representing one or more predicting factors comprise a data record of the blood glucose level of the subject at a time point.

7. The method of claim 1, wherein the historical data records representing one or more predicting factors comprise a data record of a rate of change of a blood glucose level over a specific time interval.

8. The method of claim 1, wherein the historical data records representing one or more predicting factors comprise historical data records that are observed over a prior window of time.

9. The method of claim 8, wherein the data processing engine determines the parameters based on historical data records that are received within the fixed moving time window.

10. The method of claim 8, wherein during the step of determining the parameters, the data processing engine gives less weight to historical data records that received at points further in the past with a forgetting factor configured to define how long in the past before weight becomes equal to e⁻¹.

11. The method of claim 9, wherein the fixed time window is 1 month, 3 months, 6 months, or 12 months.

12. The method of claim 1, wherein the method further comprises:
sending an alert to the subject or the subject's caregiver when the blood glucose level of the subject for the time interval of interest is outside a predetermined range.

13. The method of claim 12, wherein the method further comprises:
adjusting an insulin pump for the subject upon receiving the alert.

14. A computer-implemented method of making a therapy recommendation for an insulin pump parameter, the method comprising:
(1) receiving a blood glucose level at a first time point;
(2) receiving a rate of change of the blood glucose level at a second time point;
(3) determining an adjusted value for an insulin pump parameter based on the blood glucose level at the first time point and the rate of change of the blood glucose level at the second time point; and
(4) making a therapy recommendation for an insulin pump parameter based on the adjusted value.

15. The method of claim 14, wherein the insulin pump parameter is a basal rate for a time window.

16. The method of claim 15, wherein the basal rate in time windows is from 12:00 AM to 1:00 AM, from 1:00 AM to 2:00 AM, or from 2:00 AM to 3:00 AM.

17. The method of claim 14, wherein the adjusted value for the insulin pump parameter is determined by comparing the rate of change of the blood glucose level to a desired rate of change of the blood glucose level.

18. The method of claim 14, wherein an insulin pump parameter is modulated when the difference between the adjusted value for the insulin pump parameter and the parameter that is in use is greater than a pre-determined threshold.

19. The method of claim 18, wherein the insulin pump parameter is modulated for a portion of the difference between the adjusted value for the insulin pump parameter and the parameter that is in use, wherein the portion is ⅕, ¼, ⅓, or ½.

20. The method of claim 14, wherein the insulin pump parameter is a bolus estimation (BE).

21. The method of claim 20, wherein the bolus estimation is determined by comparing the rate of change of blood glucose level at a time point to a desired rate of change of blood glucose level at the same time point.

22. The method of claim 20, wherein the bolus estimation is determined by further taking into account insulin on board (IOB).

23. The method of claim 20, wherein the bolus estimation is determined by furthering taking into account fat content in a meal.

24. The method of claim 20, wherein the bolus estimation is a meal bolus.

25. The method of claim 24, wherein the bolus estimation is determined by further taking into account the interaction between fat content and carbohydrate content.

26. The method of claim 14, wherein the first time point and the second time point is the same time point.
